SEQUENCE LISTING (54 sequences)

SEQ ID NO: 1 - DNA, 1003 bp, Cannabis sativa
atattcttcattaatttagtctcaatatttttgtgccacgtgtttctacttttgacacgtcatcatcgttaaaatttgggtgaacaaaaagaataagtttgtgggatgtatttcctttgctttgtatagttttgatatcaatgaaaattttcttactaactaaaaaataaaaaaaaagttttaacctaaagctagcaaattatttttcaactatgcaattaatttggtgtgtactctcgaattaaaatagataaattattgaggagtcttacattagtaaatcgtttgcaaaaaataaacaaaatgcaaccgaaaggtaaatttgtaattatttttatacttcaaaagaaattttattacaacggaatagtttgggttgtcaaagttcggaaatttttttattgaattattcttttaaatatgatgaataccaaaacaagtaaaataagatcgaaatctgtaataataataataataataataataataataataataataatattaataataataatatattttcaataatactcatgcctaattatttaggtacgtacaaccatattaaataatctaaacacatgttaatcagtgacggacccagaaattttactttgtggggacttatttatcattcaaaaataattatacctattttataattattcttactagatttatttaattttatgggggctttttttacgattttgatctataattatcaaatttaaaaaattatatttaattttttaaaaggcaatatttttactagtgggggctatagcccccaaactaatacacttgggtccgtccctgatgttaataaacttaattattatctgaattacactaatattttcattaatgtttttgcctaacttaccatcatcaacatatataaatacaaggcaaggcaatgcagatcttcatcacaagaaatacatgatacatataattatttgtttagaattaattaattatataattatcaaaaatg

SEQ ID NO: 2 - protein, 623 aa, Cannabis sativa
MQCIAFHQFASSSSLPIWSSIDNRFTPKTSITSISKPKPKLKSKSNLKSRSRSSTCYPIQCTVVDNPSSTITNNSDRRSANYGPPIWSFDFVQSLPIQYKGESYTSRLNKLEKDVKRMLIGVENSLAQLELIDTIQRLGISYRFENEIISILKEKFTNNNNNPNPINYDLYATALQFRLLRQYGFEVPQEIFNNFKNHKTGEFKANISNDIMGALGLYEASFHGKKGESILEEARIFTTKCLKKYKLMSSSNNNNMTLISLLVNHALEMPLQWRITRSEAKWFIEEIYERKQDMNPTLLEFAKLDFNMLQSTYQEELKVLSRWWKDSKLGEKLPFVRDRLVECFLWQVGVRFEPQFSYFRIMDTKLYVLLTIIDDMHDIYGTLEELQLFTNALQRWDLKELDKLPDYMKTAFYFTYNFTNELAFDVLQEHGFVHIEYFKKLMVELCKHHLQEAKWFYSGYKPTLQEYVENGWLSVGGQVILMHAYFAFTNPVTKEALECLKDGHPNIVRHASIILRLADDLGTLSDELKRGDVPKSIQCYMHDTGASEDEAREHIKYLISESWKEMNNEDGNINSFFSNEFVQVCKNLGRASQFMYQYGDGHASQNNLSKERVLGLIITPIPM

SEQ ID NO: 3 - DNA, 1003 bp, Cannabis sativa
aataggcaatctgataccacatgttagaaaaaattaataataaacgcaagatctatttgtatataacatagaacacaataataagatataatgaataggtatgtgtacctgattgatccatagcaattatctcagtttgatccacagcaattattccactttgatacatatcaattactttacttcaattcgatagtgcaatactatcaactaagtcttcctccctagatctctaaatcagaagcagtttatttatctgattatcaaaaggtatacaattgtgatgagacaatatgtatttatagagttagggaggggacttagctacaaaaccctagttgggctgggcctgctcatcaaaggcttcagtcaactagaaaagcccacacttctgcttcacctagactttacatacagatgttaagcccattaataagtgatcaccaacaatatgggctaacacagcaaagaactatccaatcaacccaagttttaataaaaccaataaaacttaatgtaagcccaaataaaaagtctaacatagaaaagcatctcattgtgtttcaattcaataatagctcaccatttgaatcattcctactgtgttctggttatttgagttatttgaatattcttgttgctggtgttctggttatttgagtattattgttgctggtgtgttgctttgttgctggttctcatcattgtattactctgttttaattcaatgaagttagttttcattcatgttcatttctctctgtctttctcttctctgaaactaaaaatatcaatatgtaacgtatacacttcatctgacaatttattattattttattaatcattaaaaattatggtccccacctaatttcttttaaaacttatggtccgaagcacccccacgagcactagtagtgacttgtatatatatttgtgccataattagatgttatcggcaattaaaacttggatgagtgattcgggttagctctatccatcaatg

SEQ ID NO: 4 - protein, 613 aa, Cannabis sativa
MSSIIYSPFTSLLPLKPISSASSTATINTRLKSRFRSSILVVLRPQQRRSAKYHPTVWENKHIDSFFTPYNYELHSERLQELKQITSTSLRTTKDPCILLKLIDSIQRLGLEYHFENEIEDAVSFIYAHNDQTTSNDLFMTALRFRILRQHGLFVGSDVFDRFRGRDGKFLDSLSSNKHGILSLYEASHLGMPEENVLEEAKSFTTKRLRYFSAGKMDTTLFGKQVKQSLEVPLYWRMPRSEARNFIDLYQMDETKSVTLLELAKLDYNLVQSVHQNELKELGRWWDDLGFKKNLPFARDRVVENYLWAMGIVSEPQFSKCRIGLTKFVCILTAIDDVYDIYGSLDELELFTNAVESWDIRAIRDEFPLYLKTCYLGMLNFGNEVIDDVLQNHGLNISSYIKEEWLNLCKSYLVEARWFYNDYTPSLNEYLENSSTSVGGHAAIVHACILLLDGSIPETLLDYNFNHFHSKLIYWSSLITRLSDDLGTSKDELKRGDVKKSVECYMAEKGIWEEEEAINHIKELRRNSWKMVNKEIIIGNNCLPKIMVKMCLNMARTAQFIFQHGDGIGTSTGATKHRLASLIVKPVPIIDPCSKPINGLGDSHTTIKTKIKK

SEQ ID NO: 5 - DNA, 1016 bp, Cannabis sativa
gggtagtagtatatattccctagactcgcttgactcctgtaggtgtgtggtgatcgttcattccgcttacgaaggtattagtccatcatttttccttcttggagaagccctccttactcaacctaggggtaagggtggccgtctgacttccactctagcggtcatgacacctttcatggtcgacagtcatcccaacagtcatgacttaatttacatattcttcattaatttagtctcaatatttttgtgccacgtgtttctacttttgacacgtcatcgtcgttaaaatttgggtgaagaaaaagaataagtgggatgtatttcctttgctttgtatagttttgatatcaatgaaacttttcttactaactaaaaaataaaaaaaaaagttttaacctaaagctagcaaattatttttcaactatgcaattaattttgtgtgtactctcgaattaaaatagataaattattgaggagtcttacattagtaaatcgttagcaaaaaataaacaaaatgcaaccgaaaggtaaatttgtaattatttttatacttcaaaagaaattttattacaacggaatagtttgggttgtcaaagttcggaaatttttttattgaattattcttttaaatatgatgaataccaaaacaagtaaaataagatcgaaatctgtaatactaatactaatactaataataataataataataataataataataataataataataataataataataataataataatatattttcaatagtactcatgcctaattctttacgtcgtactttcagccatgttgaataacctaaacacatgttaataaacttaattatattatcatacttacactaatattttcattaatgtttttgcctaacttaccatcatcatcaacatatataaatacaaggcaaggcaatgcagatcttcatcacaagaaattaatacatacatataattatttgtttagaattaattaattatataattaattatcaaaaatg

SEQ ID NO: 6 - protein, 610 aa, Cannabis sativa
MQCMAFHQFAPSSSLPIWSRISRSRSSTCYPIQCTVVDNPSSTITNNSDRRSANYGPPIWSFDFIQSLPIQYKGESYTRQLNKLKKEVTRMLLGLEINSLALLELIDTLQRLGISYHFKNEINTILKKKYTDNYINNNIIITNPNYNNLYAIALEFRLLRQHGYTVPQEIFNAFKDKRGKFKTCLSDDIMGVLCLYEASFYAMKHENILEEARIFSTKCLKKYMEKMENEEEKKILLLDDNNINSNLLLINHAFELPLHWRITRSEARWFIDEIYEKKQDMNSTLFEFAKLDFNIVQSTHQEDLQHLSRWWRDCKLGGKLNFARDRLMEAFLWDVGLKFEGEFSYFRRINARLFVLITIIDDIYDVYGTLEELELFTSAVERWDVKLINELPDYMKMPFFVLHNTINEMGFDVLVQQNFVNIEYLKKSVVDLCKCYLQEAKWYYSGYQPTLEEYTELGWLSIGASVILMHAYFCFTNPITKQDLKSLQLQHHYPNIIKQACLITRLADDLGTSSDELNRGDVPKSIQCYMYDNNATEDEAREHIKFLISETWKDMNKKDEDESCLSENFVEVCKNMARTALFIYENGDGHGSQNSLSKERISTLIITPIN

SEQ ID NO: 7 - DNA, 998 bp, Cannabis sativa
tataataatagctaaaattttcaaaatttaaattttgaattttaaaattttacctaattaagtaaaaaatacgttgcctaattaaaaaatattaagaaaatttaaaaaatacgtttatatataattatatctaaatgatacactaatggaacaatagctaaatttttttcttaaagatctaaatctcaaatcttatttaaaaattttaaaaaatatataacctaataaaaaaaattaaacatacatgtattccacttaaattaccaaattgaatctaattataaaaaactaattatacgtgcattgcacgtaacatcaacctactggctactatatatactataaatacaatatacaactataatgtaaaaaataagaccataacaattcatttgcaaaaaaaatacatgacactaatacattgttttttataagtggaatgcacaaaaaagaaaatatatatgttttacttggttcttttttaaaagaaaaatgctaaaagctaggtactttaaggtaccaaatattataagatgtgacatatatatcatttctacattttaatattagatctcacatattttaatttaataaatgatataaaaaattgctaatcaattataaaatgccacattaatataatattagacatcttaaaatacaaataacaatactctttttgaaaaacaggttcgcaactgctttaaaacaaagtacacaaacgttaaattttgtttggcagattaattacattaatgaaacgtgatactcaagcaatattaattgttcaaacaatatgtgtgagctagatttgtagggaaagtacgcactacaattaaccaataactactaatgtcctaatgttgattccatccaagttaatacatgctcgtgctaattcatttatactatatatataatataattttattgtgtgtgatacagaattatacatacgccctaaactaaataagctctgttgtcatatattagccatg

SEQ ID NO: 8 - protein, 556 aa, Cannabis sativa
MSYQVLASSQNDKVSKIVRPTTTYQPSIWGERFLQYSISDQDFSYKKQRVDELKEVVRREVFLECYDNVSYVLKIVDDVQRLGLSYHFENEIEKALQHIYDNTIHQNHKDEDLHDTSTRFRLLRQHGFMVSSNIFKIFKDEQGNFKECLITDILGLLSLYEASHLSYIGENILNEALAFTTTHLHQFVKNEKTHPLSNEVLLALQRPIRKSLERLHARHYISSYENKISHNKTLLELAKLDFNLLQCLHRKELSQISRWWKEIDFVHKLPFARDRIVELYLWLLGVFHEPELSLARIISTKVIALASVADDIYDAYGTFEELELLTESINRWDLNCADQLRPECLQTFYKVLLNCYEEFESELGKEESYKVYYAREAMKRLLGAYFSEARWLHEGYFPSFDEHLKVSLISCGYTMMIVTSLIGMKDCVTKQDFEWLSKDPKIMRDCNILCRFMDDIVSHKFEQQRDHSPSTVESYMRQYGVSEQEACDELRKQVINSWKEINKAFLRPSNVPYPVLSLVLNFSRVMDLLYKDGDGYTHIGKETKNSVVALLIDQIP

SEQ ID NO: 9 - DNA, 1037 bp, Cannabis sativa
atacaactaaaaatttgaaatgagtaaaacaacaacattacaaaaaaataaaatagaacaaaacatcatgaaaacaaaaatacaaaacaacaaaatgttataaaatttaactaaaagaaactactaaaaatataattcgaaaatatattttttattattatacataaagaaatataaataatttttttaatatataaatctaattagattggatcgatttttaattagattttgagaataacatacaataatcaatccaatccaattaatatctaacttttaacatctattctaatctaattgtcattgaatatctatttttttttgtaattcgattgaattggattggttcgattaaatggaattgaatattgtccaatcctaagatgttgccctacaccactttatcttgatccttgcaatttagtaacgtatattattgctaaaaggtattactgtacttagcatcattttcagtgttatatcgttatggttataattaagtattgagtcctatataatttaagaaaataacttttaaaaaatatcgctaactagtattgagcacaagtagtgctcaataacaatactctttgataacatttcaaaaaaataaaaaaatggcaaaaccacaacgttgtaatattatatattagtacatttttcatataataattactataagaaaatagacgataatatatcgacacaacatgttatgtacaataaaatatatattgaatgaatgtggcttatataattaataatatttataataaaatatcgcaagacgtagtttattcaccgattgagggcgccgccccaagggcatatacgtcactgtactcactggtcaccaagaaaaatcaaaattctcaggttaacttcctcacatttcactatatctatctatctctatatatatgtgtacatgtccatcatcataatccacaaattacaaactctactcttgcattattacacacattctcttacttatttctctctaaatctctcctcatattatatccaaaatg

SEQ ID NO: 10 - protein, 574 aa, Cannabis sativa
MTQSGVISSSTPIFKDQPAAIVRRSGNYKPTLWDAHFFQSLQVIYTEESYGKRISELKEDVRRILEKEAENPLVKLEQINDLSRLGISYHFEDQIKTILNLTFNNNNALWKKDNLYATALHFKLLRQYGFSPVSSEVFNAFKDEKKEFKESLSKDVKGMVCLYEASFYSFRGEPILDEARDFTTKHLKQYLMTRQSQFTRVDHHDDDDHDLVKLVEHALDLPLHWRLPRLEARWFIDMYAERNYDMNPTFLDFAKLDYNFVQSAYQKELKYISRWWSGSRLTERLPFARDRVVEIFYSAVALKYEAEFGFVRTVMTKIGLLLTLMDDIYDVYGTLDELQLFLEAIERWNINELDQLPDYMKILFVAFYNNVNEISYYVLKENGIHTIKYLKKALGDLCKCYMEEAKWFHSGHIPSLEEFIENGWKSITIPLCLIYHYCLITTSITEQDMEHLLQYPTILRVSGTVFRFIDDLGTSSDELERGDNPSSIQCYMREKGVSENESREHIWNLISEGWKEINEVKASNSPYSQVFIESAIDFVRGAMEMYHKGDGFGTNQDRYLKTKVVNMFFDPIPI

SEQ ID NO: 11 - DNA, 1003 bp, Cannabis sativa
taattttaggtttttttttttattacacttcatgacagttttttttttttttacatttttacggaattttatatagaaactcattttgcaactaacgctgcaacctaaattgtaacaaaaaatcgtacagaaacccatattgcaactagccctgcaaccactttagaaacccaaaccgtaaaaaaaagttaaaaaaatagtatatgggataattcccctaatttttatatgtatataataatatatagactaattaagatttttgtccccaaattttaatatatttatgtactaaattatattttttgaacatataagaccgttaaaaatttctactgaattattgaaattgttggatgtaaggactttttgtccaattatatagtaaagaaaatctagcatgaatcaaagttcatgagatatgatttggtacgtttcaaagtttgaaggacatgatttggtagatcttaaagtctaaaaagcataatttagtacatggacaatcaatgaattagtaaaattgaatgaaattggacaaaattgcttaaatccaacaattttaatagtttaaggagaatttttaattagcaaaaaagttcagagaatataatttaaaacatatcaaataagttcagtgaataaaaatcttaattagtctaatatataataatttaatacaaattaaaatttattaaaatatattatatagaaatataatatataactcaatctattattatataacacaaaacgagtcagatatatgctttcttaattttatattataatatagatttcttattttacggtattctcttgtccataatgcacttgcaagttgaataatataaaaatattaatttgtacagtttattggttgatttttgaccactattaaaatgtatgctgtcataatgcatgcataattgcatctatatatataaatttatggagcttgtagtaattaaaactcagcatacagaagaagaatatagaaataatg

SEQ ID NO: 12 - protein, 494 aa, Cannabis sativa
MQMAGVKFISYSSLYSATFSKRPSVIREVYSLKKKKNYKLPRFDFNSGQDDLFNTGLRFRLLRHNGFPTTSDVFDKFINQKGEIEDVIMGQDTLGMLSLYEASYLAANCEESLVKAMEFTRSHLKISMPFITQKLQNQVAKALELPRHLRMAPLEARNYIDEYGKELNHCPALLDLAKLEFNELQSLHKRELTEIIRWWKQLGLVEKLGFARDRPLECFLWVVGIFPGKCYSNVRIELAKTVSILLVIDDIYDTYGSLDELHLFNHAILRWDLGAMDKLPEYMKICYMALYNTTNEIGYRVLKEHGLCITQHLKKTWLDIFDAFLTEAEWFDKKYTPTLEQYLTNGVTSGGSYMALVHSFFLIGHGITDQTISMMHPYPEIFSHSGKILRLWDDLGTAKEEQERGDVASSIDCYMKEENIESEDEARKHIKKLIRSSWIELNGELKAPSALPRSITTACFDLARTAQVIYQHGDDQSFLSVEDHVQSLFFRPCQ

SEQ ID NO: 13 - DNA, 1003 bp, Cannabis sativa
gtttttgaggtgggagttgtttctcggccgcaacaatccaccatgtttcttagtttttttttctttttgttctgggaatcgattgtttttgggtgtggattgttggttgataggttttgggggtggtttctgggcagtgagaaggtgagaagaagaagaagaagaagaaagttatggtttgaagaagaagaagaaggaaaatcaggtgggtgggtggggtttcgtgtgaagcagaaaaaagaaaaaaaaaacaagttattttatgatttgaaaatattatttttagtttttttatattattttaaattttttttattaaaattataatttggaccagtaatacttcaagacgtggcattttaaaaattaataaacggacagttatcttagggaccaacggactcacagaaaatgtgaccttggggactattaccgccaatttttgaggtttgggaataatctccatcaacccttaatttttggggacttttaccgcaattatccctattatacataatattttataacaagtttttttttattattttttattatttgatgaagtaattaaagaggagttttcaatttgttaatttttcagattagcttgaaaaaaggattagcttgaaaatacaattctataatcatacaatttcaaagcatagaaaaaaaattgttcaaaaaataagaaaagaaaattaagcataattctatattggctgctacattgcaatatacgtacgtacagtcataaaaatatgcacatggatgaactattacaattagaacagaagaaaagtaaatgataaagcttcttactcttgactaactcttattaagtactgttgattaatttgaaattttcaaatcaaaatacactataaatagctgagtacatgaaacgagttttccatcaattaaagctaactccatctcgtaatttatataattatatagtgatcgtttatatacatatatatcaccaaatcgtgatttcaaaatg

SEQ ID NO: 14 - protein, 464 aa, Cannabis sativa
MAALVSSNIHANSSSQKNGSTGSDTIRRSANFHPNIFEKFTNKEGKLDDSVSSDVEGMLSLYEASHMRIHGEPILEEAVVFTTTHLVEASKEKSQLTMSSLFLAAQVNHALRQPILKGLPRVEIQRFISLYHHDPNHSHLLLRFAKLDFNVLQKLHQKELHEISKWWNGLDFASKLPFARDRLVEGYIWPVGVYFEPKYSAARVILTKVIGVTTMIDDIYDVYGTLEELELFTDAIEKWDISCSDQLPDYMKYCYEALLKLYDEIGEELAMQGRTYRMAYAKETMKKLAQSYYVEAQWFHKKYTPTLQEYMEVALVSSTYYMLTATSFLGMGEEVSAEVFHWLMNSPKIVTASAVVCRLMDDVVSHKFEQDRGHVDSSVECYMKQYNVTEEEACKELKKQVLDAWKEMNEECMEPRDVPMSVLMRVVNLGRVIDAVYKDGDGYTHAGGIMKTFVKSLFIQTLPL

SEQ ID NO: 15 - DNA, 1003 bp, Cannabis sativa
gtcgaaaatgttctactaccatgagcggctcttatgggtattgcacaaggcccaaaaaaaataaagggcctaatatatattttttttaatgcttttcatttttttaggctaattagtaattttttttcccgaactttgacatgtaccaaatcatgccccctgaactttttttgacgttaaaaactccccctcgaactattgagattgttagatttaaggacttttgtctaattttagtaaaaaaaattctaacatggatgaaagttcaaggggcatgatttagtgcataccaaagtttaagaggcatgatttggtagatatcaaagtctggggagcatgatttaatacataaacaatcactgaaacagtaaaattgaatgaaattagacaaaagtccttaaatttaacaatctcaatagttcggggggaatttttaacggccaaaaaagttcaaggggcacgatttgatacatgtcaaagttaagggaaaaaattactaattagccttttttttaatgaacccaactttttaaagggctaacaacttatcattaatcatttctatgtcaatttccaaaatctattttcaattctattattgcttttaagcaaaaaaaaaaaaaaaaatcattgttgctatttatcatttttttcaaacctttcatattttttttttctagcatttttttgttgtattacaatcaataacaatttttataaattaattagagttttttagtattttgtgataatatttataaattatattttttttataggagcttaatttttaaaattagaacaagtcttctaaaatatttaagacgccatgtctactactaatctttaatgtgaaaaagatgagacaataaattagggacatgagactttttgttccttctgatatgtctgtctctataaaggacatgagctagacttgtaatatcatcgaaattgaaagacacaggaaaaatataaaaataaataaaagataaacattatg

SEQ ID NO: 16 - protein, 589 aa, Cannabis sativa
MDTQRKLQAEQLSCPTKSHELIFDHKSDHQRRSANYTPTIWKYDFLESLNNKYDSEEYKKRSEKLIEDVRHIIVETKDLKGMLELINTIRKLSLTYHFEDEVKKVLDKISSSDYYYNNNDIKDFLVGDDLYLAALYFRLLRLHGYHVSQGIFVGYNSVDYKKGGGTHNITSTEVKVMIEVLEASHVAFECEEMLTEAKALMEENLKIAFPDNGNKYLPKHEVVHALELPSHWRVQWFDVKWQIEAYRQGDPVTNTTTTTSLLVDLAKLNFNIVQATLQKDLRELSSWWKNVGLSEKLDFARDRLVESFMCTVGLAFQPEYKSLRKCLTKVVNFILIVDDVYDVYGSLEELRHFTNAVDRWDVRETEKLPDCMKICFQALYHTTCEIASEIETNNGCKLVLPHLKGAWTDFCKSLLMEAEWYHKGYIPSLEEYLSNAWISSSGPLLLLHSYLAMPNQTNTASSLDISKDLVYNISLIIRLCNDLGTSAAEQERGDAASSIVCYMQETKSSEEEARKHIREMIRKTWKKINKKCFSTCGSSSLSLSFIDIALNTARVAHSLYQSGDAFSAQHTDYKTHILSLLVHPLIPNK

SEQ ID NO: 17 - DNA, 1003 bp, Cannabis sativa
gactgctacatacctctgtctttgggtatatggctagatgttaagttaattaccacgtaataatttttaattggttcaagttattaactttttatatatttttctagaaaaaatagtttaaacataccaataaaaaaattacacgtgaataagggtcaggtacctacagagtttgagaaatataacttaagtattattaccacaaaaaattaaatttaagtatttatgtcacaaattactattttatatataataataataataataataatatatatatatatatacagggacggacctactcttatcaatgtgggggctatactcttatcaatgtgggggctatagctcccactagcaaaaatattacctttaaaaaaattaaatgtaatttttttaatttgataattatagaccaaaataataaaaaagcccccacaaaattaataaaactagtaagagtaattataatataagtataaatattttttaatgataaataagcccccacaagtaaaaattctaggtccgtgactaatatatacatatatattctgtagctgccgcctccaatataatttgatcgttatatatacctacttttcaaacgttgtatccacttgcatgcatgcaaagtcaaatcaataacgatcgaggaatagaacatattatttcccacatataaccactatatatatgtggcttatatatgatctttatttccaaatacatagaaagaaagtgagcaattaaatctaaaaaaaacaaaaaagaaaaatgactttaattagtagtgatgaaaaacgccctaatcttgcagagtttactccaagcatttggggagattatttcatgtcttgtgcttcaaatgatgatcactcatcccttaaagtatatatgcttattgttattataatattattatttcactgattttattaaatactattcatttatatttactagttaatttcttcatggggtttgtgttcaggaaactatg

SEQ ID NO: 18 - protein, 526 aa, Cannabis sativa
MENNKESYVKIIELKEQVKKKLLHGLHPLENPLETLEYIDDIQRLGLSYYFENEIEQVLKQFHNNNNNLHRDFGDNNLYADALRFRLLREQGYFIACEVFAKYKNEKGKFKESISSDIRGMLNLYEASQMRVRREEILDEALIFTTTHLQSLVETSQLSSPYLDLVKHALMHPIRKSFQRREARLYILLYRKLPSHEELLLTLAKLDFNLLQQLHQKELNYITRWWKEFDFKSKQSFSRDRIVECYIWNYGVYFEAQTSQIRLMMTKLISLLTIIDDSYDSYGTLEELRPFTEAWMLERWDISATDNLPEYMKVCYKKCLEFFNEIEEFTKENSYCASYIKKGLQCMVRAYSKEVQWLHNKYMPTFDEYYPIGLDNAGSEELISMAFCGMGDVVTKESMDWIFSQPQPKIIRTMSIVGRLMNDIGYHKSDRRKLSKDVVASAVECYIKQYGVTDEEAIEKLNEQVNDSWKDLNEDLLYPIAIPRPLLMRVLNLVRVNHEMYREGDGFTQPTLLKNLIDTLIINPIY

SEQ ID NO: 19 - DNA, 1003 bp, Cannabis sativa
cattcctatcttgtgactaaaaagtctgtggttaaaagtattagtcacaaaaattataatttgttgtgaataaatgtgtttttagtcacaatacttgttgtgactaaaactaaattagtgaacttgtagctaaataccgtattttagtcacaagtaactttttattgtgactaaaaatcacatttagtcacaaaaaaattatgttgtaactaaaaagttgtcactaaaagtagctattttttgtagtgtataccctagactgctacatacctctgtctttgggtatatggctaaatgttaattaaatttccatgttgtaattcttaattggtccaagtggttaacttttttttttaaaaaaaaaaaaagaaaatagtttaaataaactaataacaaaataacacgtgaaaataagggtcaggtacctacagagtttaaaaaatataacttaaatattattaccaccaaaaatttaatttaagtatttatttcacaaattattcttttatatataataataataataataattatatacatatatattctgtagctgccgcctccaatataatttgatcgttatatatacctacttttcaaacgttgtacgatttcccacttgcatgcatgcaaagtcaaatctataacatggaggaatagaacatattatttcccacatattttaactactatatatatgtggcttatatatgatctttatttccaaatatatagaaagaaagtgagcaattaaatctaaaaaaaacaaaaaagaaaaatgagtttaattagtagtgatgaaaaacgccctaatcttgcagagtttactccaagcatttggggcaaatatttcatgtcttgtgcttcaaatgatgatcactcatcccttaaagtatatatgcttatattgttattatataattattatttcacttattttattgaatactattgatcatttacatgttaatttcttcatggattttgtgttcaggaaactatg

SEQ ID NO: 20 - protein, 524 aa, Cannabis sativa
MENNKESYVKIIELKEQVKNKLLHGLHPLENPLETLEYIDDIQRLGLSYYFENEIEQVLKQFHNNNNLHHDFGDNLYADALRFRLLREQGYNSACEVFAKYKNEKGKFKESILSDIRGMLNLYEASQMRVCGEKILDEALIFTTTHLQSLVETFQLSSPYLDLVKHALMHPIRKSLQRREARLYISRYHQLPSHEKLLLTLAKLDFNLFQQLHQKELNYITRWWKEFDFKSKQSFSRDRIVECYIWNYGVYFEAQTSQIRLMMTKLISLLTIIDDSYDSYGTLEELRPFTEAWMLERWDISATENLPEYMKVCYKKFLEFFNEIEEFTKENPYCASYVKKGLQCMVRAYSKEAQWLHNKYMPTFDEYYPIGLDNAGSDELISMAFCGMGNVVTKESMDWIFSQPQPKIIRTMSIVGRLMNDIGYHKSDRRKLSKDVVASAVECYIKQYGVTDEEAIKKLNEQVNDSWKDLNEDLLYPIAIPRPLLMRVLNLVRVNHEMYREGDGFTQPTLLKNLIDTLIINPIH

SEQ ID NO: 21 - DNA, 1091 bp, Cannabis sativa
tatgtacacgtggataatatccaacttagtacttaacagctaaatttagataaaaattataagttactaatggtatcgtgattttggagggttttgctgcaaaaattgagttttgggggttaagtacaagcaaaatgaaagtattgagagttttacccgcaatgaactcatttgtttttattttctttttgttagagcatctttaatggaaaaccaaaaaagtggtgtactgctatattttaacacacttagggaaaactattactctaatggtatttttaaaagtgtgataaatttggcacatgctgaaaattgtgctaaatttggaacaagatataatattacaataaatgctacttttttatttactctaatgccactaattattttgtttcattgacttttactaaactttatattctttatttatgatattactattatttaaataataaaagaatagaaaagaataatttttaaatattcttaataaaatattaaatgactatcaatataattattttttttccttattttacatattgtacccattaaaatacaattgtaaataatagagcaaatattacacaaactcattttttatactaaatttaacacaaattttaaatcttgaatatgtatatatatatatatatataaatataatatgaagggaacataatataattaaatgaagagtcattggatagatgatcattagcttttttagatggaagagttatctctccaatccctaatggtgacttttaatttttcaatctaataaagtgatcaaactataaattaatttgaataaaaaaaaaactataaattaattaagtggaagatactttctttttttttaagaaaaaaaaagtggaagagacttaattgtgtacacatttaataacaattaataattaatattaatcattaatgtttttagcctaactgttctttatcaacatatataaatacgtacaattgcaaagtaatgcataagttttcatctcaaaaaatattttattgttattaatattgttcatatatacaaatttatatatatataaatatataattacaataaaatg

SEQ ID NO: 22 - protein, 571 aa, Cannabis sativa
MDCISAKSPSDSSVTNIVRRSANFEPSIWSFDFVQSLSSKYKGEPYTSRVKKLEEDVKRMLVEMENSLAQLELIDTLQRLGVSYRFENEINTILKEKYVNINGNINNPNYNLYATALEFRLLRQHGYAVPQETFNYFKDETGKFKTNISGDIMGVLALYEASFYEKKGESILEEARIFTTERLKNYTIMISEQNKLMINNNYDYYYNIEVVNHALELPLHRRTTRIEAKWFIDMYKKKQDMNPILLEFAKLDFNMIQSTHHEDLKHIFRWWRHTKLGEKLNFARDRLMECFLWKVGIRFEPKFSYFRTTTVKLLELITLIDDIYDVYGTLDELELFTKAIERWDVEMINELPEYMKMPYIVLHNTINEMVFEILRDQQITIKIQYLKKTWVDMCRCFLQEAKWYYSGYTPTLEEYIENGWISVGAPVLIVHAYFSHSNNNKEIFECLEHGYYPTIIRHSSIIIRLTNDLATSSEELKEVMLRRQFNVTCKKKNICEEEAREHIKFLISEAWKEMNNSESDDGLIYPISLIEDARNFARIGLFMYQHGDGHSSQDNLSKERISSFIIKPIPL

SEQ ID NO: 23 - DNA, 1003 bp, Cannabis sativa
ttgttttacatagatttgatttaactagagtttatatcttgacattgattcttattgaaggttatttaccctagtttggagaacccaagcactagctgcctagaatgtgtttctaggacagatgaagcttataaatctgcagagttcatttggatttaatttttgcattttaatttttaataatccttataaattcgtagtaaaacaactaggttttatattatgtgattgaatatcagtatttcaattagaagcatctaattagatcgatttttttagtattaattgacagcaagatagaattgataagaaccattcttccagcataaaaaagattcctcgaactccactaaatcttcacaattagctttcaaaattctttttgaattaattgtcattctaaaatatttaaaaagtaataaggtgttttttttaaaaaaaaaaactcgacatatcaattgaataaattaaaaaactaaataaattattttaaacaataattataaaaatctcttcccatttacctaatcacactaaaaacaacatttacccatattaaaaatttgttaaagattaattaaagcttagtataatatatatcctacaaattaccaatcactagctgtatgtatgaatagagtatacaagtcttttaattaattaccttgttttccacgagcctataaaatagtaaccacaaattttcaaagaaagcaaatttcaaatactcttcaaaacttgggatgtttggaccactaaataaacattaaaggcatgtacatcatgttcttccctatgaaatctccaaaaaatctatctttctgtatcatcaatcacactattatgtttatatatatatatatacacataaaactagagagggaattatttcgaaataataaagaaacaataaaataatatgtcttcgagtgataataataataatatgaatcgtcctaattctatcccattttctccaagcatttggaaagattatctcatg

SEQ ID NO: 24 - protein, 531 aa, Cannabis sativa
MSNVSNNSLMENNDDSEIVMLKKEVKKKIVELDYVENRLETLEFIDSIQRLGVSYYFENQIEVVLKNICNKFHEENNDDDDLYVVALRFRLVRQQGYYMSCDVFNKFTNNKGKFEESLCNDIRGMLSLYEASQLRVHGEKILDDALIFTTNHLESAAKTSKLSSHISNQVNHALKNPIRKSLQRRQARHYMSLYHQISSHNEHLLALAILDFNLLQKLYQKELSDLTRWWKEFDYERRQSFSRERIMDCYFWTFGVFFEAQTSHIRLLMSKLIALLTLVDDIYDNFGTFQELHLLTEAIQRWDMCLIDQLPEYIQPCYKEILNLSTEIGEFTKEKSYCLNYAKKGFQELVKGYFEEAKWLHQKHNPTLDEYMPIALVTAASPLLIAISFIGMPNVVTKDSMNWIFSHPQPKSVRTLSIVGRVLNDIGFYQWRERRTEKVDFSAVNCYIKQYGVEEEEAIQRLKEQVSDSWKDLNEECLYPNNMNIPMPLLMRVLDLVRMNNELYREGDGFTREAFIKDLIDSLIINPYIQQ

SEQ ID NO: 25 - DNA, 1003 bp, Cannabis sativa
agttatttctacaaaatttatagatctttaaattatctttccaacgccactgaaatcacctcaatccgagctctagaactccagatatgatcattttagtaaaacagtttttaatcctgcgaatttatccaaacctacgaatttttaacattaattaattaaactcactaaacatattataaaaccctaatggaccttcatattgggctttaattaaacgattacttactctgtaaaaataccataatattttactttcgtagttaatacaactttaactctctctagaaaattggtacaactactctaagcaataatatcaactcactaacaacacaaagaaacaagataattatcctcgctcaattaatatccaaaaaaaaaaattaaatactattttaatctggatattacattgggtgacatattatatcgtatcgtgttcatgtttgattttttcacactaatcaatcaacaagctcaattagcatatataagaggcaaagacatggagtccagtcgaattatgaaggttgtggcttgtctactcctctatgtcttattctcaagattcaacattcatttcacattgcttgcaaattcaattatttccccaccttgaatccttctcaattcataaacatgtgcaccgagaccactcgacggatctcatccacttgaaaattacgtataaaagttgaaaattcaattttaaaattgatgtcattttcgagttatgtcatcgtgtcttaatctatgaacctttattttttacttatttaaaatgtgagtatatatgaaaagcaccctcgattaattcgatgacacacatgcatacataaattaaaccattgtttttattcattcttctttttgcagccataaaaatcttatcttgttgattaaacatggtctacataacatgtgatcctatatatatactcttaaagagataataagttccatacatatatatatatatatatatatatattttataatg

SEQ ID NO: 26 - protein, 630 aa, Cannabis sativa
MAALVSIVSNIISFNNNNNTFIRSNHNTNIIYSNKTLLMSTNNSNIISRRSANYQPPLWQFDYVQSLSSPFKDGAYVKRVEKVKEEVRVMVKRAREEEKPLSQLELIDVLQRLGISYHFEDEINDILKHIYKNKNNNNNNNNNNNNNNVYANSLEFRLLRQHGYPVSQEIFSTCKDERGNFMVSSNDVKGMLSLYEASFYLVENEDGILEETRQTTKKYLEEYIIMIMEKQQSLLDQNNNNNDNDYDYELVSHALELPLHWRMLRLESRWFIDVYEKRLDMNPTLLTLAKLDFNIVQSIYQDDLKHVFSWWESTGMGKKLEFARDRTMVNFLWTVGVAFEPHYKNFRRMITKVNALITVIDDIYDVYGTLDELELFTNAVERWDISAMDGLPEYMKTCFLALYNFINDLPFDVLKGEEGLHIIKFLQKSWADLCKSYLREARWYYNGYTPSFEEYIENAWISISGPVILSHLYFFVVNPIKEDTLLSTCFDGYPTIIRHSSMILRLKDDMGTSTDELKRGDVPKSIQCKMYEDGISEEEARQRIKLLISETWKLINKDYINLDDDDDDGDDYSPMFYKSNNINKAFIEMCLNLGRMSHCIYQYGDGHGIQDRHTKDHVLSLLIHPIPLTQ

SEQ ID NO: 27 - DNA, 1003 bp, Cannabis sativa
ataaatttcaagaaaacaaagaacaagttacaaaaagcttaccaagagcttgagcttactaaaatcttgagaaaacagcttgaattaccaaagaaaataccaaaattcttgctgctcaaagacaggccgaaagagagagagtttgagagaaaaatggattttgcttcttttcttatattttttccaattttgtaaaataaagagtaaaatgattttatttacttatttcagccaaaactaattaatcaaaatcaataacacttcatttaattgatccacataaagacaaaacactaaaagggcaaaaagaccaaaatgcccttgcccacacaaaactaatttaaagggtactaagggtaatttaggaaattctaaatttccgaccaatcccgacattcccaatgtctaaataaactgccccgctatactaaaatactaaattgtgattctactgagtcatacaccgcgttccatgttgttgggcaccgaaaatgcaaaattatgaaatttcactatattaaatcaacataaataattatttaaatatccataaataatttttataattaaatcctaattatttgctaatttctaaatttaaactaaacggtctttacataaactgtcctacctcacttactacctcacttacatgtaggtccgcccctgcatgtgaaaattgcatattatcactaagcatgtggaaattgcatattcacactattatatataagcatgtggaaattgcatattcacactactataagcatgtgaaaactgagttccacgtatatagtacatataatacaactatatatatatatatgttgtcctatattttcaagtggattgtattatattgtttctatcaaactattatattaattaattattatacctaaccgtgtccacaaaatatcaactatatatatgttggcctatgtttgttcatttggaaataataataataataataataataataatagtagtagtaaaaatg

SEQ ID NO: 28 - protein, 560 aa, Cannabis sativa
MSPCEATIDEKRPNMPKFTPTIWGDYFMSHASSHHSSLMETMENNNKESYEKIIEMKEQVKNKLLHGLHPLENPLETLEYIDDIQRLGLSYYFENEIEQNLEQFHNNYQNLIDFGDNNLYADALCFRLLRQQDIFDKYKNENEKFKESISSDIRGMLNLYEAAQMRVHGEKILDEALIFTTTHLESSVKTCQLSSPYLDLVKHALMHPIRKSLQRREARLYISLYHQLPSHEEILLILAKLDFNLLQKLHQKELSYITRWWKEFDYKSKHSFIKDRIVECYFWVYGVFFEAETSQIRLIITKLIAILTIIDDAYDSFGTLEELEPFTQAIERWDICAIDTLPEYMKIFYMKLLEIYNEIEQFSKERSYCPSYAKKGVQSLIRAYFKEAKWLHTKYIPTLEEYMPVGIDSAGSFMLISMVFIGMGDIVTKHSMDWIFSNPQPKIIQTMAIVGRVMNDIGYHKSERKKSSGEIVASTVECYMKQYGVTGEEAIEKLSQQVKDSWKDLNEDLLNPITIPRPLLMQVLKLVRVNHEIYREGDGFTQPTLLKNLIHSLIINPIDF

SEQ ID NO: 29 - DNA, 1047 bp, Cannabis sativa
ttatacctatttgacatataataaattcgttaaaaaactctaagttaaattaatacatgtaaaataagtataaattaggggtggtaaaacgtgtcatcgtgtcgtgttcgtgtcatattttatgtgacccgctttttatttcgtgtcaagcgtgtcgacctgttttttgactcgtgtctataattatctcaaccctaacccgacctgtaaaaaatcgtgtcgtgttcgtgtcgacccactgtaactcattttgttattattgaagctataattgtagagaaaaataatagatttattaacttccattagaaaacttatatacatttatgtatatatagtttgttattaatataataaaaattaaaaaaactaaaatatttttaatttcatgtaaaaaaaattaaatttaaaattttttaacttaacattgaatcactaaatatatatttttttatataattatattattattattaattttttattttattatatttaaaaaaatcgtgtcaaacgtgtcacattcgtgttaagcgggttgtgtcgtgtttgactcatttttatttcgtgtcaagtcgtgtcgacccgttttcgatccgcgtctaaatttctcgaccctaacccgtaaaattcgtgtcgtattcgtatgccgtgtcgaaacccgttttgccactactagtataaataatatgtcctaaaataattggtagggacagaaaaagcacaccaatttagggcaacaatttttttttcagtgttaagtacttaatatagtattttaaacaataaaatatatttacgtttcgagatattagaatataaaactatcatcttgcttatacactattttatacatatatatatctctcaagtttacttatgtacatctattttttttttattttttttaaatatatagttaatagtaccagcaagttatggtgtaggacaagttgcatatataaaaatatatatatgtgctatatatatgaatgtcatttccattaattttcccaaacacttgatcattactttctacaaataatg

SEQ ID NO: 30 - protein, 588 aa, Cannabis sativa
MSNIQHQILLSLSQNNNNNEKIIIHKNVVVRPTTKFHPSIWGDRFLHYNVSQQHLVECKEERVKELVEVVRKEIMISLLSSCDNNNNDIDELMKLIDSVQRLGLSHHFETQIEQMLESVYQYYYYSTTKHDLNYYKHDLHHDSIMFRLLRQHGFKVSSSGIFEKFKDEKGNFKKSLITDVSGLLSLYEASYLSYVGESILDEALAFTTTHLKAIVANNKDHPLSHQISKALERPLRKTIERLHARFYISIYEKDASHNKVLLELAKLDFNLLQCFHKMELSEIMRWWKKHDFANKFPFARDRMVELYFWMLGIYYEPKYSRARKLLTTKISALISITDDIYDAYGTIDELELLTQAMQRRWDINFIDKLEPEYLKTYYKAMLTSYEEFEKEFTKEELYKHQYAKEEQMKKLIRAYLEEARWLNDGYLPSFDEHLKVSYVSCAYTTLIATSYVGMHDIVTHETLNWLSKDPKIVSASTLLARFMDDIGSRKFEQERNHIPSTVECYMKQYEVSEKEAIEELNKRVVNYWKEINEDFIRPTVMPFPILVRVLNVTKVLDLLYKNGDDQYTHVGKVFKESIAALLIDPIPV

SEQ ID NO: 31 - DNA, 1003 bp, Cannabis sativa
tatttgaacgatttgttttataaaaataaaatatgtgtcagtatatttagtaagtaaaaatagttgtgttacaataatgtatgttattaatagtaaaatgtagattaatcttctaattgaaaaaagttctattatctcattttatatttaataatagctacacaactatatatttatagactttcacattctttgcaaattactaataagcaccaataatattttcatattctaatatttttcaaccttctcctcttggtggtaaaattaaaaataaaactaaaaataagcacaatcatataaggtaaatattaattacagcccatttcatctttttttttattattttttttaagcccaagcccaaaaatctctaagccaacacaattaatcttcttttatattatcttttctagatattttatctatatttttcttttttaaaaaataaataaaaaaattattaatttaaaaagagattattaatatttaaaagattgtttatttaaaagattgtttaattttttggtttaattattgggtttattttttaacgggagatcaaacctaatcgttaaatttaacagaatattattttatttttaagtatattctgttaaactaagaatgttaaaccaagaaatgccgttagataggcacttttaatatataaagatattatatttatatataaaattatacatagtaactatgttatatatatagagaaaaacaaaagagttagacacgcaaacaacttgaagccaaatcgaccaaagactttgaaacaaagacacaaatattgacttgatattaatttgccgtgtttttccataatttgtccacttctaattagcttcttttgcctataaatagatatagaactttccaatattcattcaaaaacaacaacattattcattagtgttatctaaagtctttaagtatatactagctatctagtgttatattgtcatcgattgattcatcatttgccgttatg

SEQ ID NO: 32 - protein, 732 aa, Cannabis sativa
MEFQSLPQTQAKLINEIKEMMFSSLDINPYSLVSPCAYDTAWLAMIPHHNRPSKPMFEGCLSWVLNNQTEHGFWGNCDDQSGMPTLECLTATLACVVALRKWNVGSSMISKGLEFIHSSNAKRLLKEMKKEGFIPQWFAIVFPGMIELAEEVLNIQILNDQSAVVSDIFYHRQLIFQKELHNKETYLLSYLEVLPSSYFNEELIIKKLCEKGSLFQSPSATAQAFMATENSKCLHYLQTLVHKFSNNNNNNIIGVPTTYPMDKDLIKLCIINYVERLGLAEHFTIEIEQLLQQVYKNYVKCDGEFYYEKSYHSLATLELHLLKESLAFRLLRMHGYKVFPSNIYWILKNEDIKNHIESNYECFSVTMLNLYRATDLAFHGEFELDELRIFSRKLLQKSILVGARHTNPFNKLIENELSLPWMARLDHLEHRLFIEQTNEAQSSLWMGKTSFQRLSRFHNDKLVRLATLNYEFKQSIYKTELEQLTRWCKYWGLNEMGFGREKSTYCYFAVASACCSLPYDSPIRLMVAKGAIIITITDDFFDMKESLITELKTFTKAFQRWDGKELSGVSKKIFDALDNLVSEMATMYLEQQENSNHDITNWLRKIWYETICSWLTESEWSKNGIVPTMDEYLKVGMTSIATHTLLLPASCFVINPTLPVYSQLRPIQCESVTKLVMTICRLLNDLQSYEKEKEEGKPNSITVYMKNNSEVEMEEAVKTRKESLKASKVTDD

SEQ ID NO: 33 - DNA, 1071 bp, Cannabis sativa
ttataaataaactatatataaaacaaccaaaattagtttaatatatttattgaagaggtgtttttataaataaattatttcttaaaaatcaagctttaatccaatataattcaaaactatatattttttaggataagttgaagaatacaaataccctcatatcactacaagaaatgtcacttttgccagcacatttttgtacttgcaaaagttggtattgcgctggtaaaatagaatttacccatgatgtgttggtaaaagggggttggcaaatcaatgttggtattagaatttttgccagcataaaatgaaaagcgctggcaaaaaagacttttctcagggcgtaaatgtgctggtaaaagttagattttagccactgcaaatgtacgctggtaaaactttgatctctttgcccagtagctatttaaatattggtaaagttacttttaccaataaagatttaacctgtacggtaaaagtgacatttcttgtagtgtattatatttattaaaatatacctacttttgtaaatgggttgcctaatgtgctcttacactccacttaagtctagcacataattttcattaacttaaggggtataataggaacatcatctataaaaatgagtatatcttaaagaatatgaaaataaaggtaagacttaaaatatgtaaagtgggtatacaataatgtaattttttttttgggattttttataataaatttaaaaattaatgctataattaaattattcaaacaaaataatattatattttaattaagattttaaattgtaaaatattcaatacacataacatgtgaaaggcacctatatgtcacatgctagtgtgacatattccaatataaacgcctttaaaaaataataaataaataaataaataattgacttgacatagtttttctttttttggaaagaaaaatgacttgatatagtttgctgtatcaacttcttaggtagctagttttctttaatttggctataaatagctatataccttaccttttgtacatactttcattcatattgtcaattcatcatttgtcattatg

SEQ ID NO: 34 - protein, 978 aa, Cannabis sativa
MELKSLSKNQDKLIKEIKETIFSSLNINPYSLVSLNSAYEIAWIAMIPNHRQPSEPMFEGCLSWVLNNQTEHGFWGNNNESGMPTLGSLTATLACVVALKKWHVGSDMISKGLEFIHSSNAKRLLKEMKEEGFIPQWFAIIFPGMIELAEQILNIQILKDESVVVSNIFYHRQLIFQKEQHHNKEANLLSYLEVLPLSYFNKEEDYNYDIIIKKLCEEGSFFQSPSATACAFMATQNSKCLHYLQTLVHTYSNNNIINNITIVPTTYPMDEDLIKLCVINHVERLGLAEHFSMEIEQLLQHTYKNYVKHDGEFFYKKSYHSVVTLELHLLKESLAFRLLRMHGYKVFPSNICWFMKNEKIKNCIESNYESFLVTTLNLYRATDFAFHGEHELDELRTFSRKLLEKSISVGARHTNPFNKLIEHELSLSWMARLDHLEHRVYIEATDQTHDALWMGKSSQRLLSVHNDKLVCLANLNYRLKQSLFKTELEHLTRWCKEWGLSEMGFGREKSTYCYFAVASVCCSLDYNSPIRMMIAKSAILITITDDFFDMKGSIITELNTFTKAVQRWDGEGLCGVSKKIFDALDNHVKEMAITMYLDQQEKNHDDITNWLKEIWYETICSWLTESEWSKSGIVPTMDEYLKVGMTSIATHTLLIPASFLLKSGLKMQSMEYDNITKLVMIICRLLNDIQSYEKEKDEGKPNSITVYMKNNSEVEMEEAVKLLHLSCLKVFQMFFNSSNRYDSNTEILDDIAKAIYVPLKSSDDHEGLKKNLMIRLPKPLTIDPSRGKSTTVKCLLGTKMQGRYHVKATVIAEGPVLSDIQVASLMQEFTEDEIKNDVFSIPGIKSPRPYGFGSFFFQENWDLIGKDICEAISSFLQSGNLLKEINSTVITLIPKIKFPNKDLIRNYGRKVSKPNCMIKIDLQKAYDTLDWDFLEEMLIALKFPRKFSNLVLTCVKTPRYSLMFNGSLHGFFEADRDA

SEQ ID NO: 35 - protein, 527 aa, Cannabis sativa
MEYTRNIREMKNVMNSIILADDDYRERSIEALNLVNAVLRVGIDYHVQDEIKSILEREHIIFSDHISNQYFNNINQDHLYEVSLRFRLLRQGGYDVSPDVFNELMKDKKGNFNVLVEEDREGLRELFEASQVRIEGEEVLEEAEVFSGGHLKEWANLHRHTSEARSIQLTLDQPCHKSLARVTSPNFLDSFSANTTTIDQGWTWMTLLNKLVTMDFKIVQSIHQREIVLVSKWWKELGLAEELKFARDQPLKWYLWTVASLPDPSLSEERIELTKPISLVYIIDDIFDVYGTLDELTLFTDAVNNWEIKEQLPDYLKICFKALDDITNKISYVVYRKHGWNPIDSLRKSWGKLCNAFLLEAEWFGCGKLPNEEEYLKNAIVSSGVHVVLVHMFFLLGEGISMEAVNLLDNIPGLVSSTAAILRLWDDLGSAKDENQNGHDGSYVECYMKRHKECSMGEAREQVIRMIKNEWERLNKECFSSKHFPMCFRKGCLNAARMVPVMYDYDDHHRLPGLQNYINSLLSHQTI

SEQ ID NO: 36 - DNA, 1003 bp, Cannabis sativa
aaatattaataacaaaatatatatatttgaaagaataaaattaatttttttgataactaatgaaattggttggtatattttattttgtttaaacttaataaatttttaatgtttttgtgttaattaaacctaaagaaactaagatgttacttttaattttgattaccatttgataaagaaatatatacttcccacatattaaattaatcacagcaaaaagtaggttgtgatggtacaattcttgaaaccaaatcattaccaaaccaatatatagttggaaaatatttttagaattattttaaaaaaaaaataaagaaagaaagagagaaaatttgataagtatctatcaatttaaaaaagaatattgttattgaaaaaattatatatatatatatatatagcttgaaaatatatcagtgagataaataaaaaataagaagaaggagatagataaattgtttaggaaaaagatgaaagagaaagaaacaaaaaaaacggatcaaaaaaaaaggaagaaaaaaaacaaaaactaacttatatgtaaaagaggaagaaaagaacaaaacaatatactaaaaatctgaagcaaaataataggcagccgcagacgtatatacgtgtttaagaaaataagaaaattaacgttatatttcacaattaaattactagtgtggtagttgtgatatttatttcaaattttcggtaactggccgtaaatttctctttaatttagtgtgatacatccttttttaaaaaaaaaacaaaaaaaaaataaaaactatagggcctagtacctggttgcccacccttgggccaactatgtagcatgtttgatataacgcacatgtttttttgttctttgaataagtaagtacaccataaacaatattattattactatatataattatttgtatggtatacgtttaactaataaagttgtgagttgtggtttaggatataccaaaatttgtttgtgtttcatatatcgatctcagccatg

SEQ ID NO: 37 - protein, 576 aa, Cannabis sativa
MSLSGLISTTTFKEQPAIVRRSGNYKPPLWDAHFIQSLQVIYTEESYGKRINELKEDVRRILEKEAENPLVKLEQINDLSRLGISYHFEDQIKAILNLTYNNNNNALWKKNNLYATALHFKLLRQYGFNPVSSEVFNAFKDEKKEFKESLSKDVKGMVCLYEASFYSFRGEPILDEARDFTTKHLKQYLMMTRQGKTISVDHDDNNDLMVKLVEHALELPVHWRMKRLEARWFIDMYAEMSHHHHMNSTFLQLAKLDFNVVQSTYQEDLKHAVRWWKTTSLGERLPFARDRIVETFLWTVGVKFEPQFRYCRKMLTKMGQLVTSMDDIFDVYGTLDELSLFQDALERWDINTIDQLPDYMKIFFLAAYNVVNEMAYDVLKQNGILIIKYLKKTWTDLCKCYMLEANWYHSGYTPSLEEYIKNGWISIAEPLILVNLYCLITNPIKEDDMDCLLQYPTFIRISGIIARLVDDLGTSSDELKRGDNPKSIQCYMKENGICDEKNGREHIRNLISETWKEMNEARVGESPFSQAFIETAIDFVRTAMMIYQKEQDGVGTNFDHYTKDGIISLFFTSIPI

SEQ ID NO: 38 - protein, 533 aa, Cannabis sativa
QGESYTRQLNKLKKEVTRMVLGLEINSLALLELIDTLQRLGISYHFKNEINTILKKKYTDNYINNNIIITNPNYNNLYAIALEFRLLRQHGYTVPQEIFNAFKDKRGKFKTCLSDDIMGVLCLYEASFYAMKHENILEEARIFSTKCLKKYMEKMENEEEKKILLLDDNNINSNLLLINHAFELPLHWRITRSEARWFIDEIYEKKQDMNSTLFEFAKLDFNIVQSTHQEDLQHLSRWWRDCKLGGKLNFARDRLMEAFLWDVGLKFEGEFSYFRRINARLFVLITIIDDIYDVYGTLEELELFTSAVERWDVKLINELPDYMKMPFFVLHNTINEMGFDVLVQQNFVNIEYLKKSWVDLCKCYLQEAKWYYSGYQPTLEEYTELGWLSIGASVILMHAYFCFTNPITKQDLKSLQLQHHYPNIIKQACLITRLADDLGTSSDELNRGDVPKSIQCYMYDNNATEDEAREHIKFLISETWKDMNKKDEDESCLSENFVEVCKNMARTALFIYENGDGHGSQNSLSKERGDCLL

SEQ ID NO: 39 - DNA, 1003 bp, Cannabis sativa
acccaattaatgtacgtaccacggaatacattccgtgaaagtacagacaaaatcatatctcaatatatatatatatatatatacattaaagccaataaaatgtaaaagattttgaacaatacacatactttattcataaaaaaaaatgtaaaaggatggtgtagctaagagtgctacacttgctagccaaccagctggtttatgtcaagggaaccctctcgagttgttctttcaaaaaagaaaaaattatttgacaaccttcttttcgtacttcatacaaaagtgcattgcacattttttaataccactaacttccaaaatacaaatcacttttttttttgtggttgaaaataatcgtttcatagatggagataattagatcattacctaaaatatttttttatataaattttgtatgattaaatcctttcaattatcattttatttgcacttaacttttaaagttcaaattttagtggtaaaaaaaccctccaaactattcaactattaacaaatttaagatttcatctatttttcacaataaattactaacataaactacttacctacattcttatacatgtgacacatttatattggtacacttaatattattatttaaaaattataaaaaaaaaattcaaaatttataatatcttttattttataattttatttaattttaaaataataatattaggtgtatcagtataaatgtgttatgtgtataagagtgtatataagcaatctatggtgaaagttaatgggatgatactttaaatttattaacacttcaacagtttagataattttaccaccaaaatttaaacttaaagagttaagtgcaagtaaaaagacagttgaaagggtttagccgccaaaaactcttttgatatttatcacacttcaatcataggaggtaggttgttggaaacactaaccatatataaatacaaggtcgagcaaacctaacattctcatcccaaaaacacaaacaaaaatg

SEQ ID NO: 40 - protein, 179 aa, Cannabis sativa
MHCITLTHQISPLLPNICSTTNFGVFFRPKVYTNYNIINNNATKSRLSSACYPIQCAVVNSSNAIIDRRSANFEPSIWSFDYIQSLTSQYKGEPYTSRVKKLERDVKKMLVEMENSLAQLELIDTLQRLGISYRFENEINSILNKKYVNINNPNYNLYAIALQFRLLRQHGYAVPQGIY

SEQ ID NO: 41 - protein, 442 aa, Cannabis sativa
MWDWVKFIWDLKHKGIGAEEVYLYASVVVDTIWRTRNDKVHNNYIVNVKNCIDYICSSYANLHATIFPSPSACSKVSWSPPPQDWIKLNCDVKVGLDSMCSTLVVRNHLGRVVWVQTSRVDFSDALCGEVAACCLAISTAKDIGAKFVIVESNSREHEFAKKFPFARDRIVELYFWILGVYYEPKYSQARKLLTKVIALTSITDDIYDAYGTIDELQLLTQAMQRWDISYIDKLEPEYLKTYYKAMLDSYEEFEKELKKKEIYKLEYAKEEMKRMIRAYFEEARWLNQKYFPSLDEHLRVSYVTNCGNIMLIATSFVGMDNDIVTNQTLQWLSNDPKIVKASTLLSRHMNDIASRKFEQERNHIPSTVECYMKQYGVSEEEAVEELNKRVVNYWKEINEDFIRPTAVPFPILIRVLNFTKVAELIYKEDNENVYFKGMVIRA

SEQ ID NO: 42 - protein, 303 aa, Cannabis sativa
MEGENILDEAALFSAQHLEASMTHLHRYDQYQAKFVATTLQNPTHKSLSKFTAKDLFGVYPSENGYINLFKQLAKVEFTRVQSLHRMEIDKVTRWWRDIGLAKELTFARDQPVKWYIWSMACLTDPILSKQRVALTKSISFIYVIDDIFDIYSSLDELILFTQAVSSWKYSAMEKLPDSMKTCFKALDNMINESSHTIYQKRGWNPLHSLRKTDENQEGHDGSYVECYMKELGGSVEDAREEMMEKISDAWKCLNKECILRNPAFPPPFLKASLNLARLVPLMYNYDHNQRLPHLEEHIKSLL

SEQ ID NO: 43 - protein, 556 aa, Cannabis sativa; identical to SEQ ID NO: 8

SEQ ID NO: 44 - DNA, 762 bp, Cannabis sativa
aaaaattaaacatacatgtattccacttaaatcacaaaattgaacctaattataaaaaactaatattacgtgcattgcatgtaatatcaacctagtatatatactataatacaatatacaactataatgtaagaaataagactataacaattcatttgcaaaaaaaatacaaacatgacactaatacattattttttataagtggaatgcagaaaaaagaaaatatacatgttttacttggttctttttcaaaagaaaaatgctaaaagttaggtactttaagataccaaacattataagatgtgacatatatatcatctctacattttaatattaatctcacatatttttatttaataaatgatataaaaaattgctaatcaattataaaatgtcatattaatataatattagacatcttaaaatacaaataacaatactctttttgagaaacaggttcgcaactcctttaaaacaaagtacacaaacgttaaattttgtttggcagattaattacattaatgaaacgtgatactcaagcaatattaattgttcaaacaatatgtgtgagctagatttgtagggaaagtacgcacaacaattaactaataactcctaatgtcctaatgttgattccatccaagttaatacatgctcgtgctaattcatatatactatatatataatataattttattgtgtgtgatacagaattatatacgccctaaactaaataagctctgttatcatatattagccatg

SEQ ID NO: 45 - protein, 622 aa, Cannabis sativa
MQCIAFHQFASSSSLPIWSSIDNRFTPKTSITSISKPKPKLKSKSNLKSRSRSSTCYSIQCTVVDNPSSTITNNSDRRSANYGPPIWSFDFVQSLPIQYKGESYTSRLNKLEKDVKRMLIGVENSLAQLELIDTIQRLGISYRFENEIISILKEKFTNNNDNPNPNYDLYATALQFRLLRQYGFEVPQEIFNNFKNHKTGEFKANISNDIMGALGLYEASFHGKKGESILEEARIFTTKCLKKYKLMSSSNNNNMTLISLLVNHALEMPLQWRITRSEAKWFIEEIYERKQDMNPTLLEFAKLDFNMLQSTYQEELKVLSRWWKDSKLGEKLPFVRDRLVECFLWQVGVRFEPQFSYFRIMDTKLYVLLTIIDDMHDIYGTLEELQLFTNALQRWDLKELDKLPDYMKTAFYFTYNFTNELAFDVLQEHGFVHIEYFKKLMVELCKHHLQEAKWFYSGYKPTLQEYVENGWLSVGGQVILMHAYFAFTNPVTKEALECLKDGHPNIVRHASIILRLADDLGTLSDELKRGDVPKSIQCYMHDTGASEDEAREHIKYLISESWKEMNNEDGNINSFFSNEFVQVCQNLGRASQFIYQYGDGHASQNNLSKERVLGLIITPIPM

SEQ ID NO: 46 - DNA, 1001 bp, Cannabis sativa
atttggtgtgtactctcgaattaaaatagataaattattgaggagtcttacattagtaaatcgtttgcaaaaaataaacaaaatgcaaccgaaaggtaaatttgtaattatttttatacttcaaaagaaattttattacaacggaatagtttgggttgtcaaagttcggaaatttttttattgaattattcttttaaatatgatgaataccaaaacaagtaaaataagatcgaaatctgtaatactaatactaatactaataataataataatgtttatgtctctgctttctctttttctctcttgctctctagctctgggaggtggccagaaaaagctggtcttcagtagttgagaagccctagctctctctaagctaactcctttgaagttgtcatgcaaaaaatggtatgcaaatgtggatggttaatattaggcgtatgcaaccctctatatatatacacacaaaatttcatatacgcggtagtagtagacccaataagagaaaataattaaataattggattttagctaagcttgggagagtggtattaaactcattcttgtggcatgacaggtggagctttagtaggtagtgtaccataaattcattcatttcactcaaaaccaaaaatctgaggctcacgtgccttcatcttcgcgtgtaaaaaattctccattgcaaatgtccaaaagggccagagaggtgtacaccgttcactactaatctctagtagggacttgggtggactcgagtagtttctatggggccattattgtagctatagcccccaaactaatacacttgggtccgtccctgatgttaataaacttaattattatctgaattacactaatattttcattaatgtttttgcctaacttaccatcatcaacatatataaatacaaggcaaggcaatgcagatcttcatcacaagaaatacatgatacatataattatttgtttagaattaattaattatataattatcaaaaatg

SEQ ID NO: 47 - DNA, 243 bp, Artificial Sequence
  Description of Artificial Sequence Synthetic polynucleotide
atttkgtgtgtactctcgaattaaaatagataaattattgaggagtcttacattagtaaatcgttwgcaaaaaataaacaaaatgcaaccgaaaggtaaatttgtaattatttttatacttcaaaagaaattttattacaacggaatagtttgggttgtcaaagttcggaaatttttttattgaattattcttttaaatatgatgaataccaaaacaagtaaaataagatcgaaatctgtaat

SEQ ID NO: 48 - DNA, 194 bp, Artificial Sequence
  Description of Artificial Sequence Synthetic polynucleotide
cwsatgttaataaacttaattatattatcwkamttacactaatattttcattaatgtttttgcctaacttaccatcatcatcaacatatataaatacaaggcaaggcaatgcagatcttcatcacaagaaatwmatrcatacatataattatttgtttagaattaattaattatataattaattatcaaaaatg

SEQ ID NO: 49 - DNA, 274 bp, Artificial Sequence
  Description of Artificial Sequence Synthetic polynucleotide
gactgctacatacctctgtctttgggtatatggctaratgttaaktwaattwccaygtwrtaattyttaattggtycaagtkrttaactttttwtwtwwwwwwmwaraaaagaaaatagtttaaayawacyaataamaaaatwacacgtgaaaataagggtcaggtacctacagagtttraraaatataacttaartattattaccacmaaaaattwaatttaagtatttatktcacaaattaytmttttatatataataataataataataat

SEQ ID NO: 50 - DNA, 476 bp, Artificial Sequence
  Description of Artificial Sequence Synthetic polynucleotide
tatatacatatatattctgtagctgccgcctccaatataatttgatcgttatatatacctacttttcaaacgttgtaygatttcccacttgcatgcatgcaaagtcaaatcwataacgatsgaggaatagaacatattatttcccacatattttaacyactatatatatgtggcttatatatgatctttatttccaaatayatagaaagaaagtgagcaattaaatctaaaaaaaacaaaaaagaaaaatgastttaattagtagtgatgaaaaacgccctaatcttgcagagtttactccaagcatttggggmrawtatttcatgtcttgtgcttcaaatgatgatcactcatcccttaaagtatatatgcttatattgttattatawwattattatttcactkattttattraatactattsatytatatttacwwgttaatttcttcatggrktttgtgttcaggaaactatg

SEQ ID NO: 51 - DNA, 675 bp, Cannabis sativa
ttgatatcaatgaaacttttcttactaactaaaaaataaaaaaaaaagttttaacctaaagctagcaaattatttttcaactatgcaattaattttgtgtgtactctcgaattaaaatagataaattattgaggagtcttacattagtaaatcgttagcaaaaaataaacaaaatgcaaccgaaaggtaaatttgtaattatttttatacttcaaaagaaattttattacaacggaatagtttgggttgtcaaagttcggaaatttttttattgaattattcttttaaatatgatgaataccaaaacaagtaaaataagatcgaaatctgtaatactaatactaatactaataataataataataataataataataataataataataataataataataataataataataatatattttcaatagtactcatgcctaattctttacgtcgtactttcagccatgttgaataacctaaacacatgttaataaacttaattatattatcatacttacactaatattttcattaatgtttttgcctaacttaccatcatcatcaacatatataaatacaaggcaaggcaatgcagatcttcatcacaagaaattaatacatacatataattatttgtttagaattaattaattatataattaattatcaaaaatg

SEQ ID NO: 52 - DNA, 872 bp, Cannabis sativa
ttgatatcaatgaaaattttcttactaactaaaaaataaaaaaaaagttttaacctaaagctagcaaattatttttcaactatgcaattaatttggtgtgtactctcgaattaaaatagataaattattgaggagtcttacattagtaaatcgtttgcaaaaaataaacaaaatgcaaccgaaaggtaaatttgtaattatttttatacttcaaaagaaattttattacaacggaatagtttgggttgtcaaagttcggaaatttttttattgaattattcttttaaatatgatgaataccaaaacaagtaaaataagatcgaaatctgtaataataataataataataataataataataataataataatattaataataataatatattttcaataatactcatgcctaattatttaggtacgtacaaccatattaaataatctaaacacatgttaatcagtgacggacccagaaattttactttgtggggacttatttatcattcaaaaataattatacctattttataattattcttactagatttatttaattttatgggggctttttttacgattttgatctataattatcaaatttaaaaaattatatttaattttttaaaaggcaatatttttactagtgggggctatagcccccaaactaatacacttgggtccgtccctgatgttaataaacttaattattatctgaattacactaatattttcattaatgtttttgcctaacttaccatcatcaacatatataaatacaaggcaaggcaatgcagatcttcatcacaagaaatacatgatacatataattatttgtttagaattaattaattatataattatcaaaaatg

SEQ ID NO: 53 - DNA, 782 bp, Cannabis sativa
ataacctaataaaaaaaattaaacatacatgtattccacttaaattaccaaattgaatctaattataaaaaactaattatacgtgcattgcacgtaacatcaacctactggctactatatatactataaatacaatatacaactataatgtaaaaaataagaccataacaattcatttgcaaaaaaaatacatgacactaatacattgttttttataagtggaatgcacaaaaaagaaaatatatatgttttacttggttcttttttaaaagaaaaatgctaaaagctaggtactttaaggtaccaaatattataagatgtgacatatatatcatttctacattttaatattagatctcacatattttaatttaataaatgatataaaaaattgctaatcaattataaaatgccacattaatataatattagacatcttaaaatacaaataacaatactctttttgaaaaacaggttcgcaactgctttaaaacaaagtacacaaacgttaaattttgtttggcagattaattacattaatgaaacgtgatactcaagcaatattaattgttcaaacaatatgtgtgagctagatttgtagggaaagtacgcactacaattaaccaataactactaatgtcctaatgttgattccatccaagttaatacatgctcgtgctaattcatttatactatatatataatataattttattgtgtgtgatacagaattatacatacgccctaaactaaataagctctgttgtcatatattagccatg

SEQ ID NO: 54 - DNA, 787 bp, Cannabis sativa
gttgtcactaaaagtagctattttttgtagtgtataccctagactgctacatacctctgtctttgggtatatggctaaatgttaattaaatttccatgttgtaattcttaattggtccaagtggttaacttttttttttaaaaaaaaaaaaagaaaatagtttaaataaactaataacaaaataacacgtgaaaataagggtcaggtacctacagagtttaaaaaatataacttaaatattattaccaccaaaaatttaatttaagtatttatttcacaaattattcttttatatataataataataataataattatatacatatatattctgtagctgccgcctccaatataatttgatcgttatatatacctacttttcaaacgttgtacgatttcccacttgcatgcatgcaaagtcaaatctataacatggaggaatagaacatattatttcccacatattttaactactatatatatgtggcttatatatgatctttatttccaaatatatagaaagaaagtgagcaattaaatctaaaaaaaacaaaaaagaaaaatgagtttaattagtagtgatgaaaaacgccctaatcttgcagagtttactccaagcatttggggcaaatatttcatgtcttgtgcttcaaatgatgatcactcatcccttaaagtatatatgcttatattgttattatataattattatttcacttattttattgaatactattgatcatttacatgttaatttcttcatggattttgtgttcaggaaactatg

(19) United States
(12) Patent Application Publication
(10) Pub. No.: US 2022/0325294 A1
(43) Pub. Date: Oct. 13, 2022

(54) CANNABIS TERPENE SYNTHASE PROMOTERS FOR THE MANIPULATION OF TERPENE BIOSYNTHESIS IN TRICHOMES

(71) Applicant: 22nd Century Limited, LLC, Williamsville, NY (US)

(72) Inventors: Paul Rushton, Buffalo, NY (US); Sujon Sarowar, Williamsville, NY (US)

(73) Assignee: 22nd Century Limited, LLC, Williamsville, NY (US)

(21) Appl. No.: 17/620,971

(22) PCT Filed: Jun. 30, 2020
(86) PCT No.: PCT/US2020/040339

§ 371 Date: Dec. 20, 2021

Related U.S. Application Data
(60) Provisional application No. 62/869,353, filed on Jul. 1, 2019.

(51) Int. Cl. C12N 15/82; C12N 9/88

(52) U.S. Cl. CPC C12N 15/8243; C12N 15/8223; C12N 15/8205; C12N 9/88

(57) ABSTRACT

The present technology provides terpene synthase (TPS) promoters and TPS promoter consensus sequences from Cannabis, nucleotide sequences of the TPS promoters and consensus sequences, and uses of the promoters and consensus sequences for modulating the production of terpenes and other compounds in organisms The present technology also provides chimeric genes, vectors, and transgenic cells and organisms, including plant cells and plants, comprising the TPS promoters and consensus sequences. Also provided are methods for expressing nucleic acid sequences in cells and organisms using the TPS promoters and consensus sequences.

### CROSS-REFERENCE TO RELATED APPLICATION

The present application claims priority to U.S. Provisional Patent Application No. 62/869,353, filed on Jul. 1, 2019, the contents of which are hereby incorporated by reference in their entirety.

### TECHNICAL FIELD

The present technology relates generally to terpene synthase (TPS) promoters from Cannabis, nucleotide sequences of the TPS promoters, and uses of the promoters for modulating terpene biosynthesis or for modulating the production of other biochemicals in glandular trichomes in organisms. The present technology also relates to transgenic cells and organisms, including plant cells and plants, comprising the TPS promoters.

### BACKGROUND

The following description is provided to assist the understanding of the reader. None of the information provided or references cited is admitted to be prior art.

Plant trichomes are epidermal protuberances, including branched and unbranched hairs, vesicles, hooks, spines, and stinging hairs covering the leaves, bracts, and stems. There are two major classes of trichomes, which may be distinguished on the basis of their capacity to produce and secrete or store secondary metabolites, namely glandular trichomes and non-glandular trichomes. Non-glandular trichomes exhibit low metabolic activity and provide protection to the plant mainly through physical means. By contrast, glandular trichomes, which are present on the foliage of many plant species including some solanaceous species (e.g., tobacco, tomato) and also Cannabis, are highly metabolically active and accumulate metabolites, which can represent up to 10-15% of the leaf dry weight (Wagner et al., Ann. Bot. 93:3-11 (2004)). Glandular trichomes are capable of secreting (or storing) secondary metabolites as a defense mechanism.

Cannabis (Cannabis sativa L.) plants produce and accumulate a terpene-rich resin in glandular trichomes (Booth et al., 2017). Terpenes and the related terpenoids comprise a large class of biologically derived organic molecules synthesized from the condensation of the five-carbon units of isoprene. Monoterpenes (e.g., α-pinene, β-pinene, myrcene, limonene, β-ocimene, terpinolene) and sesquiterpenes (e.g., β-caryophyllene, bergamotene, farnesene, α-humulene, alloaromadendrene, δ-selinene) are important components of Cannabis resin as they are responsible both for much of the scent of Cannabis flowers and for the unique flavor qualities of Cannabis products. Other types of terpenes include diterpenes, sesterterpenes, triterpenes, sesquarterpenes, tetraterpenes, polyterpenes, and hemiterpenes. Terpenes are important compounds in the food, cosmetics, pharmaceutical and biotechnology industries. Terpenes in hop (Humulus lupulus), which is a close relative of Cannabis, are important as flavoring compounds in the brewing industry. Terpenes may also influence medicinal qualities of different Cannabis strains and varieties, and are under investigation for their potential anxiolytic, antibacterial, anti-inflammatory, sedative, and other pharmaceutical effects.

Cannabis varieties display different pharmaceutical properties as a result of their varying content of biologically active cannabinoids and terpenes. The interactions between the various cannabinoids and terpenes within the human body leads to the so-called “entourage effect,” which is the likely result of a mixture of cannabinoids and terpenes interacting with multiple different receptors within the human body, whereas a single cannabinoid or terpene may interact with only one.

Terpene biosynthesis in plants is catalyzed by terpene synthases (TPSs), which are part of a large and diverse gene family contributing to both general and specialized metabolism. The biosynthesis of terpenes involves two pathways to produce the 5-carbon isoprenoid diphosphate precursors of all terpenes, the plastidial methylerythritol phosphate (MEP) pathway and the cytosolic mevalonate (MEV) pathway. These pathways control the substrate pools available for the terpene synthases (TPSs). The plant TPS gene family has been divided into six subfamilies. Members of the a, b, c, and e/f families have previously been presented from Cannabis, including nine full length cDNAs from the hemp variety, Finola, and a total of 33 complete TPS gene models and additional partial sequences from the Purple Kush variety. However, several of these 33 genes are duplicates or are possible pseudogenes containing retrotransposon sequences.

Terpene synthase promoters from Cannabis have not been characterized for their possible efficacy in manipulating terpene biosynthesis or other biosynthetic activities in glandular trichomes. Such information may provide opportunities to select and modulate terpenes of interest to produce plant strains and varieties with desirable terpene profiles. Accordingly, there is a need to identify and characterize Cannabis TPS promoters to identify genes coding for novel activities with relevance to terpene biosynthesis and to modulate the synthesis of terpenes in organisms including transgenic plants, transgenic cells, and derivatives thereof, which allow for high-level gene expression in glandular trichomes.

### SUMMARY

Disclosed herein are terpene synthase (TPS) promoters and uses of these promoters for directing the expression of coding nucleic acid sequences in plant trichomes and other plant tissues.

In one aspect, the disclosure of the present technology provides a synthetic DNA molecule. The synthetic DNA molecule comprises a nucleotide sequence selected from the group consisting of: (a) a nucleotide sequence set forth in any one of SEQ ID NOs: 1, 3, 5, 7, 9, 11, 13, 15, 17, 19, 21, 23, 25, 27, 29, 31, 33, 36, 39, 47, 48, 49, or 50; (b) a nucleotide sequence that encodes for a polypeptide having the amino acid sequence of any one of SEQ ID NOs: 2, 4, 6, 8, 10, 12, 14, 16, 18, 20, 22, 24, 26, 28, 30, 32, 34, 35, 37, 38, 40, 41, or 42; or (c) a nucleotide sequence that is at least about 80% identical to the nucleotide sequence of (a) or (b), and which encodes a promoter having plant glandular trichome transcriptional activity. Preferably, the nucleotide sequence is operably linked to a heterologous nucleic acid. In some embodiments, the present technology provides an expression vector comprising the DNA molecule operably linked to one or more nucleic acid sequences encoding a polypeptide. In some embodiments, the present technology provides a genetically engineered host cell comprising the expression vector. In some embodiments, the cell is a Cannabis sativa cell. In some embodiments, the cell is a Nicotiana tabacum cell.

In some embodiments, the present technology provides a genetically engineered plant comprising a cell comprising a chimeric nucleic acid construct comprising the synthetic DNA molecule. In some embodiments, the plant is an N. tabacum plant. In some embodiments, the plant is a C. sativa plant. In some embodiments, the present technology provides seeds from the engineered plant, wherein the seeds comprise the chimeric nucleic acid construct.

In one aspect, the disclosure of the present technology provides a genetically engineered plant or plant cell comprising a chimeric gene integrated into its genome, the chimeric gene comprising a terpene synthase (TPS) promoter operably linked to a homologous or heterologous nucleic acid sequence. The promoter can be selected from the group consisting of: (a) a nucleotide sequence of any one of SEQ ID NOs: 1, 3, 5, 7, 9, 11, 13, 15, 17, 19, 21, 23, 25, 27, 29, 31, 33, 36, 39, 47, 48, 49, or 50; (b) a nucleotide sequence that encodes for a polypeptide having the amino acid sequence of any one of SEQ ID NOs: 2, 4, 6, 8, 10, 12, 14, 16, 18, 20, 22, 24, 26, 28, 30, 32, 34, 35, 37, 38, 40, 41, or 42; or (c) a nucleotide sequence that is at least about 80% identical to the nucleotide sequence of (a) or (b), and which encodes a promoter that has plant glandular trichome transcriptional activity. In some embodiments, the genetically engineered plant or plant cell is N. tabacum. In some embodiments, the genetically engineered plant or plant cell is C. sativa.

In one aspect, the disclosure of the present technology provides a method for expressing a polypeptide in plant trichomes, comprising first introducing into a host cell an expression vector comprising a nucleotide sequence. The nucleotide sequence is selected from the group consisting of: (i) a nucleotide sequence set forth in any one of SEQ ID NOs: 1, 3, 5, 7, 9, 11, 13, 15, 17, 19, 21, 23, 25, 27, 29, 31, 33, 36, 39, 47, 48, 49, or 50; (ii) a nucleotide sequence that encodes for a polypeptide having the amino acid sequence of any one of SEQ ID NOs: 2, 4, 6, 8, 10, 12, 14, 16, 18, 20, 22, 24, 26, 28, 30, 32, 34, 35, 37, 38, 40, 41, or 42; or (iii) a nucleotide sequence that is at least about 80% identical to the nucleotide sequence of (a) or (b), and which encodes a promoter that has plant glandular trichome transcriptional activity. Preferably, the nucleic acid sequence of (i) or (ii) is operably linked to one or more nucleic acid sequences encoding a polypeptide. Second, the method comprises growing the plant under conditions which allow for the expression of the polypeptide.

In one aspect, the disclosure of the present technology provides a method for increasing a terpene in a host plant glandular trichome. The method first comprises introducing into a host cell an expression vector comprising a nucleotide sequence. The nucleotide sequence can be selected from the group consisting of: (i) a nucleotide sequence set forth in any one of SEQ ID NOs: 1, 3, 5, 7, 9, 11, 13, 15, 17, 19, 21, 23, 25, 27, 29, 31, 33, 36, 39, 47, 48, 49, or 50; (ii) a nucleotide sequence that encodes for a polypeptide having the amino acid sequence of any one of SEQ ID NOs: 2, 4, 6, 8, 10, 12, 14, 16, 18, 20, 22, 24, 26, 28, 30, 32, 34, 35, 37, 38, 40, 41, or 42; or (iii) a nucleotide sequence that is at least about 80% identical to the nucleotide sequence of (a) or (b), and which encodes a promoter that has plant glandular trichome transcriptional activity. Preferably, the nucleic acid sequence of (i) or (ii) is operably linked to one or more nucleic acid sequences encoding an enzyme of the terpene biosynthetic pathway. Second, the method comprises growing the plant under conditions which allow for the expression of the terpene biosynthetic pathway enzyme; wherein expression of the terpene biosynthetic pathway enzyme results in the plant having an increased terpene content as compared to a control plant grown under similar conditions. In some embodiments, the terpene biosynthetic pathway enzyme is limonene synthase, squalene synthase, phytoene synthase, myrcene synthase, germacrene D synthase, a-farnesene synthase, or geranyllinalool synthase. In some embodiments, the method further comprises providing the plant with isopentenyl diphosphate (IPP), dimethyl allyl diphosphate (DMAPP), or geranyl pyrophosphate (GPP). In some embodiments, the present technology provides a genetically-engineered plant produced by the method, wherein the plant has increased terpene content relative to a control plant.

In one aspect, the disclosure of the present technology provides a genetically engineered plant or plant cell comprising a chimeric gene integrated into its genome, the chimeric gene comprising a terpene synthase (TPS) promoter operably linked to a homologous or heterologous nucleic acid sequence, wherein the promoter is selected from the group consisting of: (a) a nucleotide sequence of any one of SEQ ID NOs: 44 or 46; (b) a nucleotide sequence that encodes for a polypeptide having the amino acid sequence of any one of SEQ ID NOs: 43 or 45; and (c) a nucleotide sequence that is at least about 80% identical to the nucleotide sequence of (a) or (b), and which encodes a promoter that has plant glandular trichome transcriptional activity. In some embodiments, the plant contains glandular trichomes. In some embodiments, the plant is an N. tabacum plant. In some embodiments, the plant is a C. sativa plant.

In one aspect, the disclosure of the present technology provides a method for expressing a polypeptide in plant trichomes, comprising: (a) introducing into a host cell an expression vector comprising a nucleotide sequence selected from the group consisting of: (i) a nucleotide sequence set forth in any one of SEQ ID NOs:44 or 46; (ii) a nucleotide sequence that encodes for a polypeptide having the amino acid sequence of any one of SEQ ID NOs: 43 or 45; and (iii) a nucleotide sequence that is at least about 80% identical to the nucleotide sequence of (a) or (b), and which encodes a promoter that has plant glandular trichome transcriptional activity; wherein the nucleic acid sequence of (i) or (ii) is operably linked to one or more nucleic acid sequences encoding a polypeptide; and (b) growing the plant under conditions which allow for the expression of the polypeptide.

In one aspect, the disclosure of the present technology provides a method for increasing a terpene in a host plant glandular trichome, comprising: (a) introducing into a host cell an expression vector comprising a nucleotide sequence selected from the group consisting of: (i) a nucleotide sequence set forth in any one of SEQ ID NOs: 44 or 46; (ii) a nucleotide sequence that encodes for a polypeptide having the amino acid sequence of any one of SEQ ID NOs: 43 or 45; and (iii) a nucleotide sequence that is at least about 80% identical to the nucleotide sequence of (a) or (b), and which encodes a promoter that has plant glandular trichome transcriptional activity; wherein the nucleic acid sequence of (i) or (ii) is operably linked to one or more nucleic acid sequences encoding an enzyme of the terpene biosynthetic pathway; and (b) growing the plant under conditions which allow for the expression of the terpene biosynthetic pathway enzyme; wherein expression of the terpene biosynthetic pathway enzyme results in the plant having an increased terpene content relative to a control plant grown under similar conditions. In some embodiments, the terpene biosynthetic pathway enzyme is limonene synthase, squalene synthase, phytoene synthase, myrcene synthase, germacrene D synthase, α-farnesene synthase, or geranyllinalool synthase. In some embodiments, the method further comprises providing the plant with isopentenyl diphosphate (IPP), dimethyl allyl diphosphate (DMAPP), or geranyl pyrophosphate (GPP). In some embodiments, the disclosure of the present technology relates to a genetically-engineered plant produced by the method, wherein the plant has increased terpene content relative to a control plant.

Both the foregoing summary and the following description of the drawings and detailed description are exemplary and explanatory. They are intended to provide further details of the invention, but are not to be construed as limiting. Other objects, advantages, and novel features will be readily apparent to those skilled in the art from the following detailed description of the invention.

### BRIEF DESCRIPTION OF THE DRAWINGS

FIG. 1 is a schematic depicting the molecular phyologenetic analysis of the TPS proteins from the CBDRx genome together with published TPS proteins from across the plant kingdom. CBDRx proteins are designated by filled circles. The evolutionary history was inferred by using the Maximum Likelihood method (Jones et al., 1992). The tree with the highest log likelihood is shown. The tree is drawn to scale, with branch lengths measured in the number of substitutions per site. Evolutionary analyses were conducted in MEGA6 (Tamura et al., 2013).

FIGS. 2A-2B are images showing the CsTPS1/35PK (Group 1; FIG. 2A) and CsTPS4FN (Group 2; FIG. 2B) promoters direct expression in trichomes.

FIGS. 3A-3B are dendrograms showing the evolutionary relationship of Cannabis TPS promoters. FIG. 3A: The evolutionary history was inferred using the Neighbor-Joining method (Saitou and Nei, 1987). The optimal tree is shown and is drawn to scale, with branch lengths in the same units as those of the evolutionary distances used to infer the phylogenetic tree. Red circles denote TPS promoters from the CBDRx genome (red circles include, in order of appearance from top to bottom, TPS9Rx, TPS10Rx, TPS19Rx), TPS15Rx, TPS6Rx, TPS8Rx, TPS12Rx, TPS14Rx, TPS16Rx, TPS17Rx, TPS11Rx, TPS5Rx, TPS7Rx, TPS4Rx, TPS3Rx, TPS1Rx, TPS13Rx, TPS2Rx), green from the Finola genome (green circle appears for TPS4FN), and blue from the Purple Kush genome (blue circle appears for TPS1/35PK). A red open circle denotes a potential promoter from a pseudogene (red open circle appears for TPS21Rx Pseudogene). Four clades of promoters are boxed. Numbers indicate bootstrap values from 100 iterations. FIG. 3B: The evolutionary history was inferred using the Neighbor-Joining method (Saitou and Nei, 1987). The optimal tree is shown and is drawn to scale, with branch lengths in the same units as those of the evolutionary distances used to infer the phylogenetic tree. Red circles denote TPS proteins from the CBDRx genome (red circles include, in order of appearance from top to bottom, TPS1Rx, TPS3Rx, TPS11Rx, TPS13Rx, TPS19Rx, TPS5Rx, TPS8Rx, TPS2Rx, TPS6Rx, TPS18Rx, TPS4Rx, TPS15Rx, TPS7Rx, TPS12Rx, TPS14Rx, TPS9Rx, TPS10Rx, TPS17Rx, and TPS16Rx), green from the Finola genome (green circle appears for CsTPS4FN), and blue from the Purple Kush genome (blue circle appears for CsTPSK1/35). A red open circle denotes the truncated n-terminus from a pseudogene (red open circle appears for TPS21Rx Pseudogene). Four clades of proteins that correspond to the clades of promoters in FIG. 2A are boxed. Numbers indicate bootstrap values from 100 iterations.

FIG. 4 shows the Group 1 TPS promoter comparison and consensus sequence. The analysis was performed using Pro-coffee.

FIG. 5 shows the Group 2 TPS promoter comparison and consensus sequence. The analysis was performed using Pro-coffee.

FIG. 6 shows the Group 3 TPS promoter comparison and consensus sequence. The analysis was performed using Pro-coffee.

FIG. 7 shows the Group 4 TPS promoter comparison and consensus sequence. The analysis was performed using Pro-coffee.

### DETAILED DESCRIPTION

### I. Introduction

The present technology relates to the discovery of nucleic acid sequences for twenty-three genes in the CBDRx Cannabis genome. Of these, nineteen are full-length terpene synthase (TPS) promoter genes and four pseudogenes in the CBDRx geneome. The TPS genes and pseudogenes have been given arbitrary names and assigned a putative enzymatic activity and are listed in Table 1.

TABLE 1
CBDRx TPS Genes and Pseudogenes.

| | | | Possible |
|---|---|---|---|
| | | CBDRx Genome | Enzymatic |
| Name | CBDRx Genome Model | Position | Activity |
| TPS1CBDRx | novel_model_4025/6/7/8/9/30_5bd9a17a.2.5bd9b139 | Chr: 5 2425683-2431371 | Limonene synthase |
| TPS2CBDRx | evm.model.10.1522 | Chr: 10 53239717- | Squalene/phytoene |
| | | 53242880 | synthase |
| TPS3CBDRx | novel_gene_2177_5bd9a17a4032 | Chr: 5 2517081-2518425 | Myrcene synthase |
| TPS4CBDRx | evm.model.08.692 | Chr: 8 21354581- | Terpene synhtase |
| | | 21365793 | |
| TPS5CBDRx | evm.model.07.850/novel_model_6189_5bd9a17a | Chr: 7 23662609- | Terpene synthase |
| | | 23663214 | |
| TPS6CBDRx | evm.model.10.1532 | Chr: 10 53364320- | Squalene/phytoene |
| | | 53368431 | synthase |
| TPS7CBDRx | evm.model.02.1601 | Chr: 2 64561719- | Germacrene D |
| | | 64564617 | synthase |
| TPS8CBDRx | evm.model.02.2842 | Chr: 2 93293500- | Alpha-fame sene |
| | | 93297744 | synthase |
| TPS9CBDRx | evm.model.08.1595 | Chr: 8 69292828- | Squalene/phytoene |
| | | 69296133 | synthase |
| TPS10CBDRx | evm.model.08.1592 | Chr: 8 69213914- | Squalene/phytoene |
| | | 69218317 | synthase |
| TPS11CBDRx | evm.TU.ctgX15.1 | Chr: ctgX15 192516- | Terpene synthase |
| | | 198550 | |
| TPS12CBDRx | evm.model.08.1948 | Chr: 8 77979993- | Squalene/phytoene |
| | | 77983481 | synthase |
| TPS13CBDRx | novel_model_3987_5bd9a17a | Chr:5 1323376-1331221 | Terpene synthase |
| TPS14CBDRx | evm.model.08.1938 | Chr: 8 77744894- | Squalene/phytoene |
| | | 77749438 | synthase |
| TPS15CBDRx | evm.model.08.1886 | Chr: 8 76723032- | Terpene synthase |
| | | 76736166 | |
| TPS16CBDRx | evm.model.09.33 | Chr: 9 540755-545833 | Geranyllinalool |
| | | | synthase |
| TPS17CBDRx | evm.model.09.34 | Chr: 9 550266-564739 | Geranyllinalool |
| | | | synthase |
| TPS18CBDRx | evm.model.02.2840 | Chr: 2 93260668- | Squalene/phytoene |
| | | 93263532 | synthase |
| TPS19CBDRx | evm.TU.07.849 | Chr: 7 23494397- | Terpene synthase |
| | | 23503748 | |
| TPS20CBDRx | evm.model.05.137 | Chr:5 2468324-2483517 | Terpene synthase |
| TPS21CBDRx | novel_model_4020_5bd9a17a | Chr:5 2023571-2034571 | Terpene synthase |
| TPS22CBDRx | evm.model.08.1884 | Chr: 8 76633181- | Terpene synthase |
| | | 76645767 | |
| TPS23CBDRx | 1912_5bd9a17a | Chr: 2 93267649- | Squalene/phytoene |
| | | 93285072 | synthase |

The nucleic acid and corresponding amino acid sequences for each promoter have been determined, as detailed in Table 2 below.

TABLE 2
CBDRx TPS Nucleic Acid and Amino Acid Sequences.

| | Nucleic Acid Sequence | Amino Acid Sequence of |
|---|---|---|
| Promoter | of the Promoter | the Promoter Polypeptide |
| TPS1CBDRx | SEQ ID NO: 1 | SEQ ID NO: 2 |
| TPS2CBDRx | SEQ ID NO: 3 | SEQ ID NO: 4 |
| TPS3CBDRx | SEQ ID NO: 5 | SEQ ID NO: 6 |
| TPS4CBDRx | SEQ ID NO: 7 | SEQ ID NO: 8 |
| TPS5CBDRx | SEQ ID NO: 9 | SEQ ID NO: 10 |
| TPS6CBDRx | SEQ ID NO: 11 | SEQ ID NO: 12 |
| TPS7CBDRx | SEQ ID NO: 13 | SEQ ID NO: 14 |
| TPS8CBDRx | SEQ ID NO: 15 | SEQ ID NO: 16 |
| TPS9CBDRx | SEQ ID NO: 17 | SEQ ID NO: 18 |
| TPS10CBDRx | SEQ ID NO: 19 | SEQ ID NO: 20 |
| TPS11CBDRx | SEQ ID NO: 21 | SEQ ID NO: 22 |
| TPS12CBDRx | SEQ ID NO: 23 | SEQ ID NO: 24 |
| TPS13CBDRx | SEQ ID NO: 25 | SEQ ID NO: 26 |
| TPS14CBDRx | SEQ ID NO: 27 | SEQ ID NO: 28 |
| TPS15CBDRx | SEQ ID NO: 29 | SEQ ID NO: 30 |
| TPS16CBDRx | SEQ ID NO: 31 | SEQ ID NO: 32 |
| TPS17CBDRx | SEQ ID NO: 33 | SEQ ID NO: 34 |
| TPS18CBDRx | — | SEQ ID NO: 35 |
| TPS19CBDRx | SEQ ID NO: 36 | SEQ ID NO: 37 |
| TPS20CBDRx | — | SEQ ID NO: 38 |
| TPS21CBDRx | SEQ ID NO: 39 | SEQ ID NO: 40 |
| TPS22CBDRx | — | SEQ ID NO: 41 |
| TPS23CBDRx | — | SEQ ID NO: 42 |

The TPS promoters described herein are not trichome specific, as they exhibit expression in vascular tissue. Terpenes have not been shown to be cytotoxic and their expression in other tissues outside of glandular trichomes is not expected to have deleterious consequences on plant development and physiology. Accordingly, the TPS promoters described herein are useful tools for manipulating terpenes in trichomes (their main tissue of production) regardless of their expression in other plant tissues.

Accordingly, in some embodiments, the present technology provides previously undiscovered Cannabis terpene synthase (TPS) promoters or biologically active fragments thereof that may be used to genetically manipulate the synthesis of terpenes (e.g., monoterpenes such as α-pinene, β-pinene, myrcene, limonene, β-ocimene, and terpinolene, and sesquiterpenes such as β-caryophyllene, bergamotene, farnesene, α-humulene, alloaromadendrene, and δ-selinene), or other biochemicals in host plants, such as C. sativa, plants of the family Solanaceae, and other plant families and species.

### II. Genetic Engineering of Host Cells and Organisms Using Cannabis Terpene Synthase Promoters

A. Cannabis Terpene Synthase (TPS) Promoters

Terpene synthase (TPS) promoters that direct high-level expression in glandular trichomes have the potential to be useful tools in manipulating terpene biosynthesis not only in Cannabis plants but also in other plants such as tobacco, tomato, or basil. Use of these TPS promoters to make novel varieties with different combinations of terpenes and cannabinoids (e.g., altering the entourage effect) may lead to new Cannabis-based products in the medicinal and food and beverage industries. Additionally, manipulation of terpene content (or other biologically active compounds) in other plant species using these Cannabis TPS promoters may lead to novel products in the wider food, cosmetics, pharmaceutical and biotechnology industries.

Until recently, genome sequences of Cannabis varieties were relatively poor. For example, it was impossible to resolve the linkage of cannabidiolic and tetrahydrocannabinolic acid synthase gene clusters which are associated with transposable elements (Grassa et al., 2018). However, a complete chromosome assembly and an ultra-high-density linkage map of the high CBDA variety, CBDRx, has recently been made available (Grassa et al., 2018).

As described herein, this improved genome sequence data was used to: (1) identify all the potential TPS genes and pseudogenes in the CBDRx Cannabis genome; (2) identify and test TPS promoters in tobacco for glandular trichome expression; and (3) determine promoter sequences that could be used to manipulate terpene biosynthesis in Cannabis and other plants.

As described in the experimental examples, using BLAST searches and Hidden Markov Models, nineteen apparently full length TPS genes and four pseudogenes were identified in the CBDRx genome. Arbitrary names were assigned to all TPS genes from the CBDRx variety because there is no strict one-to-one correspondence to the published sequences in Finola or Purple Kush due to gene duplication and deletion (Table 1). The four pseudogenes were also numbered as they may correspond to functional genes in other varieties.

The disclosure of the present technology relates to the identification of twenty-three promoters, which are capable of regulating transcription of coding nucleic acid sequences operably linked thereto in glandular trichome cells and other plant tissues (e.g., vascular tissue).

Accordingly, the present technology provides an isolated polynucleotide having a nucleic acid sequence that is at least about 50%, about 55%, about 60%, about 65%, about 70%, about 75%, about 80%, about 85%, about 90%, about 91%, about 92%, about 93%, about 94%, about 95%, about 96%, about 97%, about 98%, about 99%, or about 100% identical to a nucleic acid sequence described in any of SEQ ID NOs: 1, 3, 5, 7, 9, 11, 13, 15, 17, 19, 21, 23, 25, 27, 29, 31, 33, 36, or 39, or to a nucleic acid sequence encoding a polypeptide described in any of SEQ ID NOs: 2, 4, 6, 8, 10, 12, 14, 16, 18, 20, 22, 24, 26, 28, 30, 32, 34, 35, 37, 38, 40, 41, or 42 wherein the nucleic acid sequence is capable of regulating transcription of coding nucleic acid sequences operably linked thereto in glandular trichome cells or other plant tissues (e.g., vascular tissue). Differences between two nucleic acid sequences may occur at the 5′ or 3′ terminal positions of the reference nucleotide sequence or anywhere between those terminal positions, interspersed either individually among nucleotides in the reference sequence or in one or more contiguous groups within the reference sequence.

The present technology also includes biologically active “variants” of SEQ ID NOs: 1, 3, 5, 7, 9, 11, 13, 15, 17, 19, 21, 23, 25, 27, 29, 31, 33, 36, or 39, or of nucleic acid sequences encoding a polypeptide described in any of SEQ ID NOs: 2, 4, 6, 8, 10, 12, 14, 16, 18, 20, 22, 24, 26, 28, 30, 32, 34, 35, 37, 38, 40, 41, or 42, with one or more bases deleted, substituted, inserted, or added, wherein the nucleic acid sequence is capable of regulating transcription of coding nucleic acid sequences operably linked thereto in glandular trichome cells or other plant tissues (e.g., vascular tissue). Variants of SEQ ID NOs: 1, 3, 5, 7, 9, 11, 13, 15, 17, 19, 21, 23, 25, 27, 29, 31, 33, 36, or 39, or of nucleic acid sequences encoding a polypeptide described in any of SEQ ID NOs: 2, 4, 6, 8, 10, 12, 14, 16, 18, 20, 22, 24, 26, 28, 30, 32, 34, 35, 37, 38, 40, 41, or 42, include nucleic acid sequences comprising at least about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or more nucleic acid sequence identity to SEQ ID NOs: 1, 3, 5, 7, 9, 11, 13, 15, 17, 19, 21, 23, 25, 27, 29, 31, 33, 36, or 39, or to nucleic acid sequences encoding a polypeptide described in any of SEQ ID NOs: 2, 4, 6, 8, 10, 12, 14, 16, 18, 20, 22, 24, 26, 28, 30, 32, 34, 35, 37, 38, 40, 41, or 42, and which are active in glandular trichomes and other plant tissues (e.g., vascular tissue).

In some embodiments of the present technology, the polynucleotides (promoters) are modified to create variations in the molecule sequences such as to enhance their promoting activities, using methods known in the art, such as PCR-based DNA modification, or standard mutagenesis techniques, or by chemically synthesizing the modified polynucleotides.

Accordingly, the sequences set forth in SEQ ID NOs: 1, 3, 5, 7, 9, 11, 13, 15, 17, 19, 21, 23, 25, 27, 29, 31, 33, 36, or 39, or nucleic acid sequences encoding a polypeptide described in any of SEQ ID NOs: 2, 4, 6, 8, 10, 12, 14, 16, 18, 20, 22, 24, 26, 28, 30, 32, 34, 35, 37, 38, 40, 41, or 42, may be truncated or deleted and still retain the capacity of directing the transcription of an operably linked nucleic acid sequence in glandular trichomes and other plant tissues (e.g., vascular tissue). The minimal length of a promoter region can be determined by systematically removing sequences from the 5′ and 3′-ends of the isolated polynucleotide by standard techniques known in the art, including but not limited to removal of restriction enzyme fragments or digestion with nucleases.

In one embodiment, a truncated polypeptide variant is at least about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 35, about 40, about 45, about 50, about 55, about 60, about 65, about 70, about 75, about 80, about 85, about 90, about 95, or about 100 contiguous amino acids in length. In other embodiments, the truncated polypeptide is truncated by about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 35, about 40, about 45, or about 50 contiguous amino acids.

TPS promoters of the present technology may be used for modulating the expression of terpenes or other biochemicals.

TPS promoters of the present technology may also be used for expressing a nucleic acid that will decrease or inhibit expression of a native gene in the plant. Such nucleic acids may encode antisense nucleic acids, ribozymes, sense suppression agents, or other products that inhibit expression of a native gene.

The TPS promoters of the present technology may also be used to express proteins or peptides in “molecular farming” applications. Such proteins or peptides include but are not limited to industrial enzymes, antibodies, therapeutic agents, and nutritional products.

In some embodiments, novel hybrid promoters can be designed or engineered by a number of methods. Many promoters contain upstream sequences that activate, enhance, or define the strength and/or specificity of the promoter. See, e.g., Atchison, Ann. Rev. Cell Biol. 4:127 (1988). T-DNA genes, for example contain “TATA” boxes defining the site of transcription initiation and other upstream elements located upstream of the transcription initiation site modulate transcription levels.

B. Consensus Sequences Driving Strong Trichome Expression

In some embodiments, the disclosure of the present technology also relates to the identification of TPS promoter consensus nucleic acid sequences and molecules that may be sufficient for directing strong trichome expression of coding nucleic acid sequences operably linked thereto.

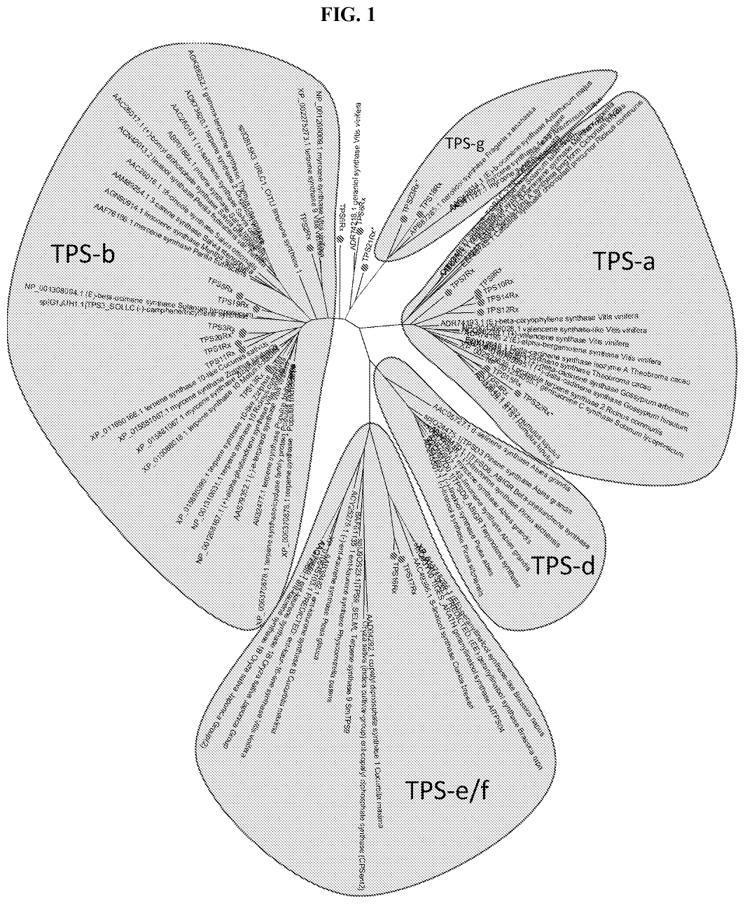

The amino acid sequences of the TPS genes were used in a combined phylogenetic tree using TPSs from across the plant kingdom (FIG. 1). The majority of the Cannabis TPS genes fall into the TPS-a and TPS-b subfamilies. TPS16CBDRx and TPS17CBDRx are members of the TPS-e/f family and are the first reported members of TPSs in this family from Cannabis. The two proteins are most closely related to geranyllinalool synthases.

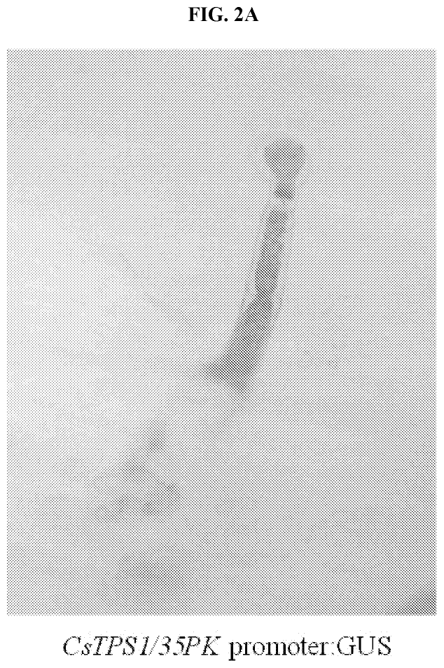

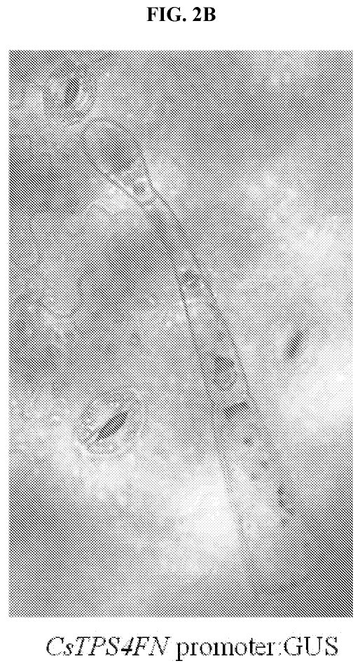

One TPS-a promoter (from the Finola TPS gene TPS4FN) and one TPS-b promoter (from the Purple Kush gene TPS1/35PK) were chosen at random and tested for the ability to drive significant expression of the GUS reporter gene in tobacco glandular trichomes. Neither promoter has previously been characterized functionally, and the published DNA sequences of TPS1PK and TPS35PK revealed them to be the same gene (KY624372, DQ839404.1, and KY624375). FIGS. 2A and 2B show that both promoters direct significant levels of gene expression in tobacco glandular trichomes and the two promoters can therefore be used to manipulate terpene biosynthesis, or the biosynthesis of other biochemicals, in glandular trichomes from plants.

Both of the tested promoters also show expression in vascular tissue, suggesting that some terpene biosynthesis may also occur there. The trichomes and vascular tissue were the only tissues that showed high level expression.

There is not a one-to-one correspondence between TPS genes from different varieties of Cannabis. In many cases, there are transposon sequences adjacent to TPS sequences and often transposon sequences appear responsible for the conversion of genes into pseudogenes. For this reason, the present inventors sought to find similarities between promoter sequences, both within the CBDRx TPS gene family and also similarities to the TPS4FN and TPS1/35 promoters, so that common promoter domains can be identified.

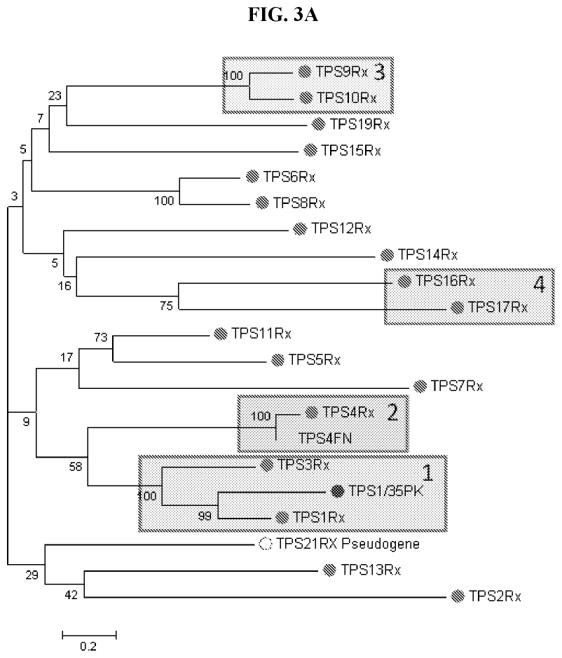

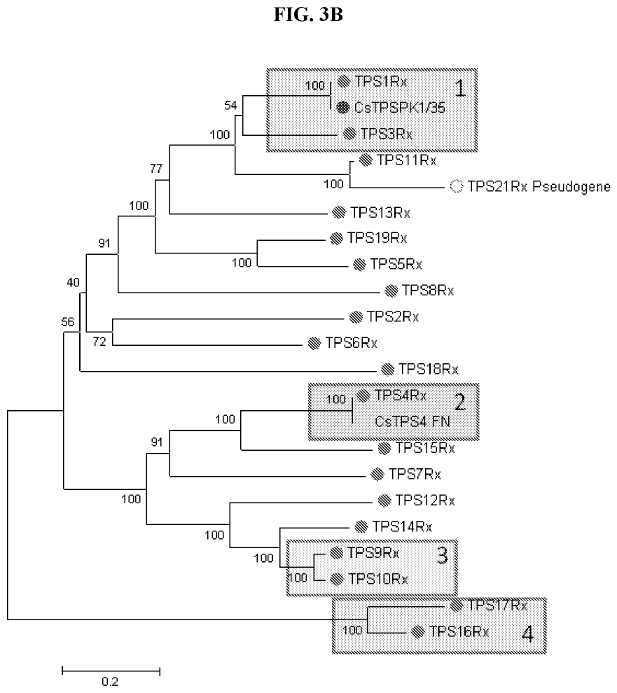

FIGS. 3A and 3B show two phylogentic trees, the first based on promoter sequences and the second on amino acid sequences. Genes that cluster together both at the amino acid level and the less conserved promoter DNA level are liable to encode closely related genes and also show similar regulation due to similar promoters.

FIGS. 3A and 3B show four such groups (named 1-4). Group 1 contains the TPS-b subfamily genes TPS1Rx, TPS3Rx, and TPS1/35PK. The Pro-coffee alignment tool for homologous promoter regions was used to compare the three promoters and to derive a consensus sequence.

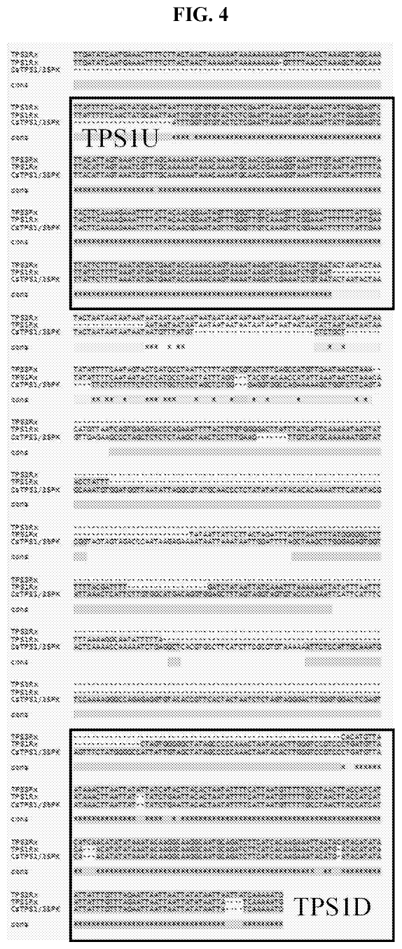

The three promoters show two highly conserved promoter regions separated by an area that shows little sequence conservation between the three promoters (FIG. 4). The two conserved promoter domains have been named TPS1U (terpene synthase Glade 1 upstream; SEQ ID NO: 47) and TPS1D (terpene synthase Glade 1 downstream; SEQ ID NO: 48) (see Table 2). Given the similarities in these genes, it is likely that the strong trichome expression activity resides in one, or both, of these two domains and that these domains are a feature of similar TPS genes in many Cannabis varieties.

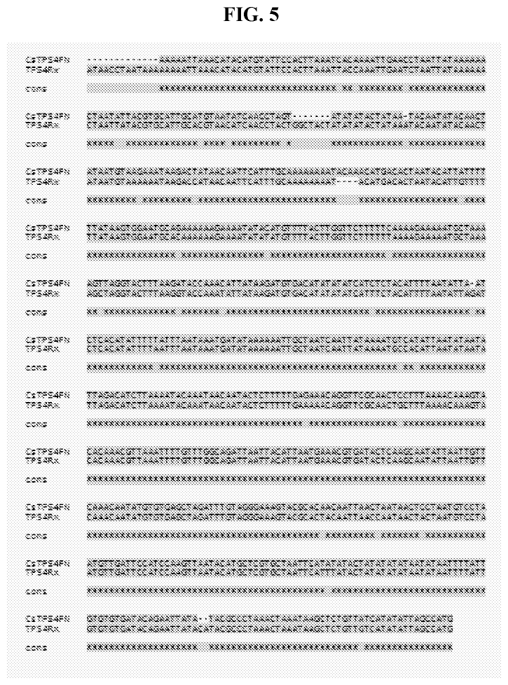

Group 2 contains the TPS-a subfamily genes TPS4Rx and TPS4FN. The Pro-coffee alignment tool for homologous promoter regions shows that the promoter regions are almost identical, and it is therefore likely that similar promoters that drive high level expression in glandular trichomes are present in many Cannabis varieties (FIG. 5).

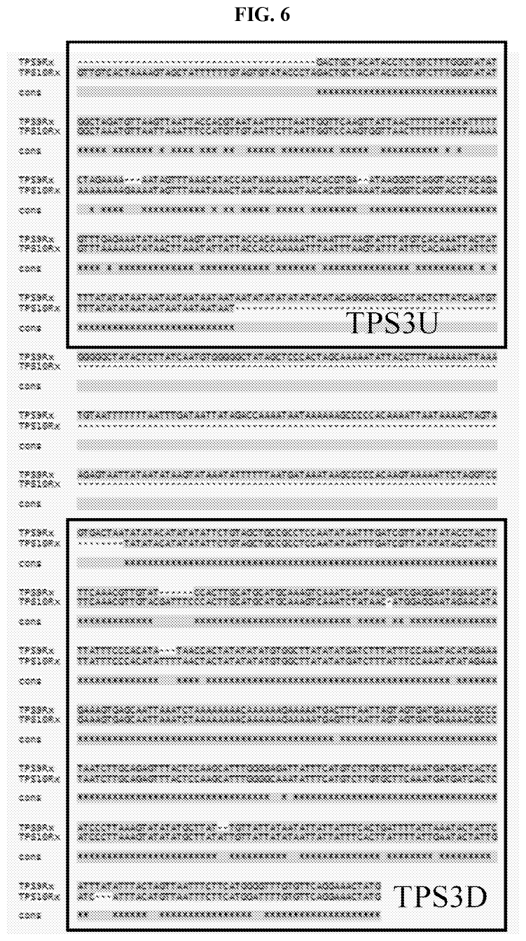

Group 3 contains promoters only from the CBDRx genome. It contains the TPS-a subfamily genes TPS9Rx and TPS10Rx. Similar to the situation in the Group 1 promoters, the promoters show two highly conserved promoter regions separated by an area that shows little sequence conservation (FIG. 6). The two conserved promoter domains are named TPS3U (terpene synthase Glade 3 upstream; SEQ ID NO: 49) and TPS3D (terpene synthase Glade 3 downstream; SEQ ID NO: 50) (see Table 2). Given the similarities in these genes, it is again likely that the strong trichome expression activity resides in one, or both, of these two domains and that these domains are a feature of similar TPS genes in many Cannabis varieties.

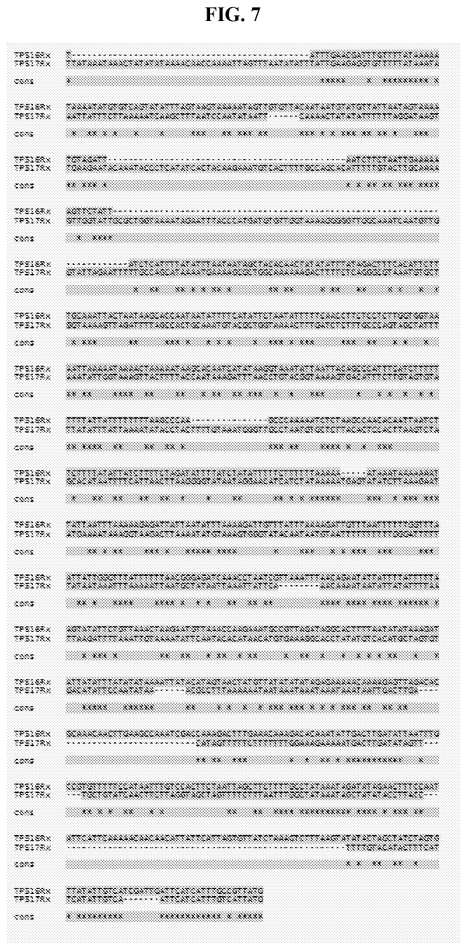

By contrast, the two Group 4 promoters from the TPS-e/f genes TPS16Rx and TPS17Rx show no appreciable similarity to each other (FIG. 7). TPS16Rx and TPS17Rx are the first reported TPS-e/f genes from Cannabis and although they cluster together in the phylogenetic tree (FIG. 1), they are dissimilar enough to show no appreciable similarity in promoter sequence.

Cannabis TPS promoter consensus sequences that are likely to drive strong trichome expression activity are shown below in Table 3.

TABLE 3
Cannabis TPS promoter consensus sequences.

```
TPS1D
ATGTTAATAAACTTAATT(AT)TATC A/T T/G A C/A
TTACACTAATATTTTCATTAATGTTTTTGCCTAACTTACC
ATCATCA (TCA)ACATATATAAATACAAGGCAAGGCAAT
GCAGATCTTCATCACAAGAAAT T/A A/C AT
A/G ATACATATAATTATTTGTTTAGAATTAATTAATTAT
ATAATTA (ATTA) TCAAAAATG (SEQ ID NO: 48)
Where X/Y represents two alternative bases
and (XYZ) indicates an insertion.
TPS1U
ATTT T/G GTGTGTACTCTCGAATTAAAATAGATAAATTAT
TGAGGAGTCTTACATTAGTAAATCGTT A/T
GCAAAAAATAAACAAAATGCAACCGAAAGGTAAATTTGTAAT
TATTTTTATACTTCAAAAGAAATTTTATTACAACGGAATAGTT
TGGGTTGTCAAAGTTCGGAAATTTTTTTATTGAATTATTCTTT
TAAATATGATGAATACCAAAACAAGTAAAATAAGATCGAAATC
TGTAAT
(SEQ ID NO: 47)
Where X/Y represents two alternative bases
and (XYZ) indicates an insertion.
TPS3D
TATATACATATATATTCTGTAGCTGCCGCCTCCAATATAATTT
GATCGTTATATATACCTACTTTTCAAACGTTGTA T/C (GATTTC)
CCACTTGCATGCATGCAAAGTCAAATC A/T ATAAC (G) AT C/G
GAGGAATAGAACATATTATTTCCCACATA (TTT) TAAC C/T
ACTATATATATGTGGCTTATATATGATCTTTATTTCCAAATA C/T
ATAGAAAGAAAGTGAGCAATTAAATCTAAAAAAAACAAAAAAGAAA
AATGA C/G
TTTAATTAGTAGTGATGAAAAACGCCCTAATCTTGCAGAGTTTACTCC
AAGCATTTGGGG A/C G/A A T/A
TATTTCATGTCTTGTGCTTCAAATGATGATCACTCATCCCTTAAAGTA
TATATGCTTAT (AT) TGTTATTATA A/T T/A ATTATTATTTCACT
  G/T ATTTTATT A/G AAT AC TAT T C/G AT T/C (TAT)
ATTTAC T/A A/T
GTTAATTTCTTCATGG G/A G/T TTTGTGTTCAGGAAACT [symbol]
(SEQ ID NO: 50)
Where X/Y represents two alternative bases and
(XYZ) indicates an insertion.
TPS3U
GACTGCTACATACCTCTGTCTTTGGGTATATGGCTA G/A ATGTTAA
  G/T T T/A AATT A/T CCA C/T GT A/T A/G TAATT T/C
TTAATTGGT T/C CAAGT T/G A/G TTAACTTTTT A/T T A/T
T A/T T/A T/A T/A T/A T/A C/A T/A A G/A AAAA (GA)
AATAGTTTAAA C/T A T/A AC C/T AATAA A/C AAAAT T/A
ACACGTGA (AA) ATAAGGGTCAGGTACCTACAGAGTTT G/A A G/A
AAATATAACTTAA G/A TATTATTACCAC A/C AAAAATT A/T
AATTTAAGTATTTAT G/T TCACAAATTA C/T T A/C
TTTTATATATAATAATAATAATAATAAT (SEQ ID NO: 49)
Where X/Y represents two alternative bases and
(XYZ) indicates an insertion.
```

Without wishing to be bound by theory, it is believed that the sequences shown in Table 3 (TPS1D, TPS1U, TPS3D, TSP3U) are responsible for the strong glandular trichome expression of Cannabis TPS promoters.

C. Nucleic Acid Constructs

In some embodiments, the Cannabis terpene synthase (TPS) promoter sequences and TPS1D, TPS1U, TPS3D, TSP3U consensus sequences of the present technology, or biologically active fragments thereof, can be incorporated into nucleic acid constructs, such as expression constructs (i.e., expression vectors), which can be introduced and replicate in a host cell, such as plant glandular trichome cell. Such nucleic acid constructs may include a heterologous nucleic acid operably linked to any of the TPS promoter sequences or consensus sequences of the present technology. Thus, in some embodiments, the present technology provides the use of any of the TPS promoters or consensus sequences set forth in SEQ ID NOs: 1, 3, 5, 7, 9, 11, 13, 15, 17, 19, 21, 23, 25, 27, 29, 31, 33, 36, 39, 47, 48, 49, or 50, or of nucleic acid sequences encoding a polypeptide described in any of SEQ ID NOs: 2, 4, 6, 8, 10, 12, 14, 16, 18, 20, 22, 24, 26, 28, 30, 32, 34, 35, 37, 38, 40, 41, or 42, or biologically active fragments thereof, for the expression of homologous or heterologous nucleic acid sequences in a recombinant cell or organism, such as a plant cell or plant. In some embodiments, this use comprises operably linking any of the TPS promoters or consensus sequences set forth in SEQ ID NOs: 1, 3, 5, 7, 9, 11, 13, 15, 17, 19, 21, 23, 25, 27, 29, 31, 33, 36, 39, 47, 48, 49, or 50, or of nucleic acid sequences encoding a polypeptide described in any of SEQ ID NOs: 2, 4, 6, 8, 10, 12, 14, 16, 18, 20, 22, 24, 26, 28, 30, 32, 34, 35, 37, 38, 40, 41, or 42, or biologically active fragments thereof, to a homologous or heterologous nucleic acid sequence to form a nucleic acid construct and transforming a host, such as a plant or plant cell. In some embodiments, various genes that encode enzymes involved in biosynthetic pathways for the production of terpenes or other biochemicals can be suitable as transgenes that can be operably linked to a TPS promoter or consensus sequence of the present technology. In some embodiments, the nucleic acid constructs of the present technology can be used to modulate the expression of terpenes or other compounds in glandular trichome cells.

In some embodiments, an expression vector comprises a TPS promoter or consensus sequence comprising a nucleic acid sequence selected from the group consisting of SEQ ID NOs: 1, 3, 5, 7, 9, 11, 13, 15, 17, 19, 21, 23, 25, 27, 29, 31, 33, 36, 39, 47, 48, 49, or 50, or a nucleic acid sequence encoding a polypeptide described in any of SEQ ID NOs: 2, 4, 6, 8, 10, 12, 14, 16, 18, 20, 22, 24, 26, 28, 30, 32, 34, 35, 37, 38, 40, 41, or 42, or a biologically active fragment thereof, operably linked to the cDNA encoding one or more polypeptides of interest (e.g., enzymes involved in the terpene biosynthesis pathway, such as limonene synthase, squalene synthase, phytoene synthase, myrcene synthase, germacrene D synthase, α-farnesene synthase, geranyllinalool synthase) for expression in a glandular trichome or other plant tissue. In another embodiment, a plant cell line comprises an expression vector comprising a TPS promoter or consensus sequence comprising a nucleic acid sequence selected from the group consisting of SEQ ID NOs: 1, 3, 5, 7, 9, 11, 13, 15, 17, 19, 21, 23, 25, 27, 29, 31, 33, 36, 39, 47, 48, 49, or 50, or a nucleic acid sequence encoding a polypeptide selected from the group consisting of SEQ ID NOs: 2, 4, 6, 8, 10, 12, 14, 16, 18, 20, 22, 24, 26, 28, 30, 32, 34, 35, 37, 38, 40, 41, or 42, or a biologically active fragment thereof, operably linked to the cDNA encoding one or more polypeptides of interest (e.g., enzymes involved in the terpene biosynthesis pathway, such as limonene synthase, squalene synthase, phytoene synthase, myrcene synthase, germacrene D synthase, α-farnesene synthase, geranyllinalool synthase) for expression in a glandular trichome or other plant tissue. In another embodiment, a transgenic plant comprises an expression vector comprising a TPS promoter or consensus sequence comprising a nucleic acid sequence selected from the group consisting of SEQ ID NOs: 1, 3, 5, 7, 9, 11, 13, 15, 17, 19, 21, 23, 25, 27, 29, 31, 33, 36, 39, 47, 48, 49, or 50, or a nucleic acid sequence encoding a polypeptide selected from the group consisting of SEQ ID NOs: 2, 4, 6, 8, 10, 12, 14, 16, 18, 20, 22, 24, 26, 28, 30, 32, 34, 35, 37, 38, 40, 41, or 42, or a biologically active fragment thereof, operably linked to the cDNA encoding one or more polypeptides of interest (e.g., enzymes involved in the terpene biosynthesis pathway, such as limonene synthase, squalene synthase, phytoene synthase, myrcene synthase, germacrene D synthase, α-farnesene synthase, geranyllinalool synthase) for expression in a glandular trichome or other plant tissue. In another embodiment, methods for genetically modulating the production of terpenes are provided, comprising: introducing an expression vector comprising a TPS promoter or consensus sequence comprising a nucleic acid sequence selected from the group consisting of SEQ ID NOs: 11, 3, 5, 7, 9, 11, 13, 15, 17, 19, 21, 23, 25, 27, 29, 31, 33, 36, 39, 47, 48, 49, or 50, or a nucleic acid sequence encoding a polypeptide selected from the group consisting of SEQ ID NOs: 2, 4, 6, 8, 10, 12, 14, 16, 18, 20, 22, 24, 26, 28, 30, 32, 34, 35, 37, 38, 40, 41, or 42, or a biologically active fragment thereof, operably linked to the cDNA encoding one or more polypeptides of interest (e.g., enzymes involved in the terpene biosynthesis pathway, such as limonene synthase, squalene synthase, phytoene synthase, myrcene synthase, germacrene D synthase, α-farnesene synthase, geranyllinalool synthase) for expression in a glandular trichome or other plant tissue.

In another embodiment, an expression vector comprises one or more TPS promoters or consensus sequences comprising a nucleic acid sequence selected from the group consisting of SEQ ID NOs: 1, 3, 5, 7, 9, 11, 13, 15, 17, 19, 21, 23, 25, 27, 29, 31, 33, 36, 39, 47, 48, 49, or 50, or a nucleic acid sequence encoding a polypeptide selected from the group consisting of SEQ ID NOs: 2, 4, 6, 8, 10, 12, 14, 16, 18, 20, 22, 24, 26, 28, 30, 32, 34, 35, 37, 38, 40, 41, or 42, or a biologically active fragment thereof, operably linked to cDNA encoding one or more polypeptides of interest (e.g., enzymes involved in the terpene biosynthesis pathway, such as limonene synthase, squalene synthase, phytoene synthase, myrcene synthase, germacrene D synthase, α-farnesene synthase, geranyllinalool synthase) for expression in glandular trichomes or other plant tissues. In another embodiment, a plant cell line comprises one or more TPS promoters or consensus sequences comprising a nucleic acid sequence selected from the group consisting of SEQ ID NOs: 1, 3, 5, 7, 9, 11, 13, 15, 17, 19, 21, 23, 25, 27, 29, 31, 33, 33, 36, 39, 47, 48, 49, or 50, or a nucleic acid sequence encoding a polypeptide selected from the group consisting of SEQ ID NOs: 2, 4, 6, 8, 10, 12, 14, 16, 18, 20, 22, 24, 26, 28, 30, 32, 34, 35, 37, 38, 40, 41, or 42, or a biologically active fragment thereof, operably linked to cDNA encoding one or more polypeptides of interest (e.g., enzymes involved in the terpene biosynthesis pathway, such as limonene synthase, squalene synthase, phytoene synthase, myrcene synthase, germacrene D synthase, α-farnesene synthase, geranyllinalool synthase) for expression in glandular trichomes or other plant tissue. In another embodiment, a transgenic plant comprises one or more TPS promoters or consensus sequences comprising a nucleic acid sequence selected from the group consisting of SEQ ID NOs: 1, 3, 5, 7, 9, 11, 13, 15, 17, 19, 21, 23, 25, 27, 29, 31, 33, 36, 39, 47, 48, 49, or 50, or a nucleic acid sequence encoding a polypeptide selected from the group consisting of SEQ ID NOs: 2, 4, 6, 8, 10, 12, 14, 16, 18, 20, 22, 24, 26, 28, 30, 32, 34, 35, 37, 38, 40, 41, or 42, or a biologically active fragment thereof, operably linked to cDNA encoding one or more polypeptides of interest (e.g., enzymes involved in the terpene biosynthesis pathway, such as limonene synthase, squalene synthase, phytoene synthase, myrcene synthase, germacrene D synthase, α-farnesene synthase, geranyllinalool synthase) for expression in glandular trichomes or other plant tissue. In another embodiment, methods for genetically modulating the production level of terpenes are provided, comprising introducing into a host cell an expression vector comprising one or more TPS promoters or consensus sequences, comprising a nucleic acid sequence selected from the group consisting of SEQ ID NOs: 1, 3, 5, 7, 9, 11, 13, 15, 17, 19, 21, 23, 25, 27, 29, 31, 33, 36, 39, 47, 48, 49, or 50, or a nucleic acid sequence encoding a polypeptide selected from the group consisting of SEQ ID NOs: 2, 4, 6, 8, 10, 12, 14, 16, 18, 20, 22, 24, 26, 28, 30, 32, 34, 35, 37, 38, 40, 41, or 42, or a biologically active fragment thereof, operably linked to cDNA encoding one or more polypeptides of interest (e.g., enzymes involved in the terpene biosynthesis pathway, such as limonene synthase, squalene synthase, phytoene synthase, myrcene synthase, germacrene D synthase, α-farnesene synthase, geranyllinalool synthase) for expression in glandular trichomes or other plant tissues.

Constructs may be comprised within a vector, such as an expression vector adapted for expression in an appropriate host (plant) cell. It will be appreciated that any vector which is capable of producing a plant comprising the introduced DNA sequence will be sufficient.

Suitable vectors are well known to those skilled in the art and are described in general technical references such as Pouwels et al., Cloning Vectors, A Laboratory Manual, Elsevier, Amsterdam (1986). Vectors for plant transformation have been described (see, e.g., Schardl et al., Gene 61:1-14 (1987)). In some embodiments, the nucleic acid construct is a plasmid vector, or a binary vector. Examples of suitable vectors include the Ti plasmid vectors.

Recombinant nucleic acid constructs (e.g., expression vectors) capable of introducing nucleotide sequences or chimeric genes under the control of a TPS promoter or consensus sequence may be made using standard techniques generally known in the art. To generate a chimeric gene, an expression vector generally comprises, operably linked in the 5′ to 3′ direction, a TPS promoter sequence or consensus sequence that directs the transcription of a downstream homologous or heterologous nucleic acid sequence, and optionally followed by a 3′ untranslated nucleic acid region (3′-UTR) that encodes a polyadenylation signal which functions in plant cells to cause the termination of transcription and the addition of polyadenylate nucleotides to the 3′ end of the mRNA encoding the protein. The homologous or heterologous nucleic acid sequence may be a sequence encoding a protein or peptide or it may be a sequence that is transcribed into an active RNA molecule, such as a sense and/or antisense RNA suitable for silencing a gene or gene family in the host cell or organism. Expression vectors also generally contain a selectable marker. Typical 5′to 3′ regulatory sequences include a transcription initiation site, a ribosome binding site, an RNA processing signal, a transcription termination site, and/or polyadenylation signal.

In some embodiments, the expression vectors of the present technology may contain termination sequences, which are positioned downstream of the nucleic acid molecules of the present technology, such that transcription of mRNA is terminated, and polyA sequences added. Exemplary terminators include Agrobacterium tumefaciens nopaline synthase terminator (Tnos), Agrobacterium tumefaciens mannopine synthase terminator (Tmas), and the CaMV 35S terminator (T35S). Termination regions include the pea ribulose bisphosphate carboxylase small subunit termination region (TrbcS) or the Tnos termination region. The expression vector also may contain enhancers, start codons, splicing signal sequences, and targeting sequences.

In some embodiments, the expression vectors of the present technology may contain a selection marker by which transformed cells can be identified in culture. The marker may be associated with the heterologous nucleic acid molecule, i.e., the gene operably linked to a promoter. As used herein, the term “marker” refers to a gene encoding a trait or a phenotype that permits the selection of, or the screening for, a plant or cell containing the marker. In plants, for example, the marker gene will encode antibiotic or herbicide resistance. This allows for selection of transformed cells from among cells that are not transformed or transfected.

Examples of suitable selectable markers include but are not limited to adenosine deaminase, dihydrofolate reductase, hygromycin-B-phosphotransferase, thymidine kinase, xanthine-guanine phospho-ribosyltransferase, glyphosate and glufosinate resistance, and amino-glycoside 3′-O-phosphotransferase (kanamycin, neomycin and G418 resistance). These markers may include resistance to G418, hygromycin, bleomycin, kanamycin, and gentamicin. The construct may also contain the selectable marker gene bar that confers resistance to herbicidal phosphinothricin analogs like ammonium gluphosinate. See, e.g., Thompson et al., EMBO J., 9:2519-23 (1987)). Other suitable selection markers known in the art may also be used.

Visible markers such as green florescent protein (GFP) may be used. Methods for identifying or selecting transformed plants based on the control of cell division have also been described. See, e.g., WO 2000/052168 and WO 2001/059086.

Replication sequences, of bacterial or viral origin, may also be included to allow the vector to be cloned in a bacterial or phage host. Preferably, a broad host range prokaryotic origin of replication is used. A selectable marker for bacteria may be included to allow selection of bacterial cells bearing the desired construct. Suitable prokaryotic selectable markers also include resistance to antibiotics such as kanamycin or tetracycline.

Other nucleic acid sequences encoding additional functions may also be present in the vector, as is known in the art. For example, when Agrobacterium is the host, T-DNA sequences may be included to facilitate the subsequent transfer to and incorporation into plant chromosomes.

Whether a nucleic acid sequence of present technology or biologically active fragment thereof is capable of conferring transcription in glandular trichomes and whether the activity is “strong,” can be determined using various methods. Qualitative methods (e.g., histological GUS (β-glucuronidase) staining) are used to determine the spatio-temporal activity of the TPS promoter or consensus sequence (i.e., whether the TPS promoter or consensus sequence is active in a certain tissue or organ (e.g., glandular trichomes, or under certain environmental/developmental conditions). Quantitative methods (e.g., fluorometric GUS assays) also quantify the level of activity compared to controls. Suitable controls include, but are not limited to, plants transformed with empty vectors (negative controls) or transformed with constructs comprising other promoters, such as the Arabidopsis CER6 promoter, which is active in the epidermis and trichomes of Nicotiana tabacum.

To test or quantify the activity of a TPS promoter or consensus sequence of the present technology, a nucleic acid sequence as set forth in SEQ ID NOs: 1, 3, 5, 7, 9, 11, 13, 15, 17, 19, 21, 23, 25, 27, 29, 31, 33, 33, 36, 39, 47, 48, 49, or 50, or a nucleic acid sequence encoding a polypeptide as set forth in SEQ ID NOs: 2, 4, 6, 8, 10, 12, 14, 16, 18, 20, 22, 24, 26, 28, 30, 32, 34, 35, 37, 38, 40, 41, or 42, or biologically active fragments thereof, may be operably linked to a known nucleic acid sequence (e.g., a reporter gene such as gusA, or any gene encoding a specific protein) and may be used to transform a plant cell using known methods. The activity of the TPS promoter or consensus sequence can, for example, be assayed (and optionally quantified) by detecting the level of RNA transcripts of the downstream nucleic acid sequence in host cells, e.g., glandular trichome cells, by quantitative RT-PCR or other PCR-based methods. Alternatively, the reporter protein or activity of the reporter protein may be assayed and quantified, by, for example a fluorometric GUS assay if the reporter gene is the gus gene.

In some embodiments, the promoters of the present technology can be used to drive expression of a heterologous nucleic acid of interest in glandular trichome cells or other plant cells. The heterologous nucleic acid can encode any man-made recombinant or naturally occurring or protein.

D. Host Plants and Cells and Plant Regeneration

The nucleic acid construct of the present technology can be utilized to transform a host cell, such as a plant cell. In some embodiments, the nucleic acid construct of the present technology is used to transform at least a portion of the cells of a plant. These expression vectors can be transiently introduced into host plant cells or stably integrated into the genomes of host plant cells to generate transgenic plants by various methods known to persons skilled in the art.

Methods for introducing nucleic acid constructs into a cell or plant are well known in the art. Suitable methods for introducing nucleic acid constructs (e.g., expression vectors) into plant glandular trichomes or other plant cells to generate transgenic plants include, but are not limited to, Agrobacterium-mediated transformation, particle gun delivery, microinjection, electroporation, polyethylene glycol-assisted protoplast transformation, and liposome-mediated transformation. Methods for transforming dicots primarily use Agrobacterium tumefaciens.

Agrobacterium rhizogenes may be used to produce transgenic hairy roots cultures of plants, including Cannabis and tobacco, as described, for example, by Guillon et al., Curr. Opin. Plant Biol. 9:341-6 (2006). “Tobacco hairy roots” refers to tobacco roots that have T-DNA from an Ri plasmid of Agrobacterium rhizogenes integrated in the genome and grow in culture without supplementation of auxin and other phytohormones.

Additionally, plants may be transformed by Rhizobium, Sinorhizobium, or Mesorhizobium transformation. (Broothaerts et al., Nature, 433: 629-633 (2005)).

After transformation of the plant cells or plant, those plant cells or plants into which the desired DNA has been incorporated may be selected by such methods as antibiotic resistance, herbicide resistance, tolerance to amino-acid analogues or using phenotypic markers.

The transgenic plants can be used in a conventional plant breeding scheme, such as crossing, selfing, or backcrossing, to produce additional transgenic plants containing the transgene.

Suitable host cells include plant cells, such as glandular trichome cells. Any plant may be a suitable host, including monocotyledonous plants or dicotyledonous plants, such as, for example, maize/corn (Zea species, e.g., Z. mays, Z. diploperennis (chapule), Zea luxurians (Guatemalan teosinte), Zea mays subsp. huehuetenangensis (San Antonio Huista teosinte), Z. mays subsp. mexicana (Mexican teosinte), Z. mays subsp. parviglumis (Balsas teosinte), Z. perennis (perennial teosinte) and Z. ramosa, wheat (Triticum species), barley (e.g., Hordeum vulgare), oat (e.g., Avena sativa), sorghum (Sorghum bicolor), rye (Secale cereale), soybean (Glycine spp, e.g., G. max), cotton (Gossypium species, e.g., G. hirsutum, G. barbadense), Brassica spp. (e.g., B. napus, B. juncea, B. oleracea, B. rapa, etc.), sunflower (Helianthus annus), tobacco (Nicotiana species), alfalfa (Medicago sativa), rice (Oryza species, e.g., O. sativa indica cultivar-group or japonica cultivar-group), forage grasses, pearl millet (Pennisetum species. e.g., P. glaucum), tree species, vegetable species, such as Lycopersicon ssp (recently reclassified as belonging to the genus Solanum), e.g., tomato (L. esculentum, syn. Solanum lycopersicum) such as e.g., cherry tomato, var. cerasiforme or current tomato, var. pimpinellifolium) or tree tomato (S. betaceum, syn. Cyphomandra betaceae), potato (Solanum tuberosum) and other Solanum species, such as eggplant (Solanum melongena), pepino (S. muricatum), cocona (S. sessiliflorum) and naranjilla (S. quitoense); peppers (Capsicum annuum, Capsicum frutescens), pea (e.g., Pisum sativum), bean (e.g., Phaseolus species), carrot (Daucus carona), Lactuca species (such as Lactuca sativa, Lactuca indica, Lactuca perennis), cucumber (Cucumis sativus), melon (Cucumis melo), zucchini (Cucurbita pepo), squash (Cucurbita maxima, Cucurbita pepo, Cucurbita mixta), pumpkin (Cucurbita pepo), watermelon (Citrullus lanatus syn. Citrullus vulgaris), fleshy fruit species (grapes, peaches, plums, strawberry, mango, melon), ornamental species (e.g., Rose, Petunia, Chrysanthemum, Lily, Tulip, Gerbera species), woody trees (e.g., species of Populus, Salix, Quercus, Eucalyptus), fibre species e.g., flax (Linum usitatissimum), and hemp (Cannabis sativa). In some embodiments, the plant is Cannabis sativa. In some embodiments, the plant is Nicotiana tabacum.

Thus, in some embodiments, the present technology contemplates the use of the TPS promoters and/or consensus sequences comprising the nucleic acid sequences set forth in SEQ ID NOs: 1, 3, 5, 7, 9, 11, 13, 15, 17, 19, 21, 23, 25, 27, 29, 31, 33, 33, 36, 39, 47, 48, 49, or 50, or a nucleic acid sequence encoding a polypeptide set forth in SEQ ID NOs: 2, 4, 6, 8, 10, 12, 14, 16, 18, 20, 22, 24, 26, 28, 30, 32, 34, 35, 37, 38, 40, 41, or 42, or biologically active fragments thereof, to genetically manipulate the synthesis of terpenes or other molecules in host plants, such as C. sativa, plants of the family Solanaceae, such as N. tabacum, and other plant families and species.

The present technology also contemplates cell culture systems (e.g., plant cell cultures, bacterial or fungal cell cultures, human or mammalian cell cultures, insect cell cultures) comprising genetically engineered cells transformed with the nucleic acid molecules described herein. In some embodiments, a cell culture comprising cells comprising a TPS promoter or consensus sequence of the present technology is provided.

Various assays may be used to determine whether a plant cell shows a change in gene expression, for example, Northern blotting or quantitative reverse transcriptase PCR (RT-PCR). Whole transgenic plants may be regenerated from the transformed cell by conventional methods. Such transgenic plants may be propagated and self-pollinated to produce homozygous lines. Such plants produce seeds containing the genes for the introduced trait and can be grown to produce plants that will produce the selected phenotype.

To enhance the expression and/or accumulation of a molecule of interest in glandular trichome cells and/or to facilitate purification of the molecule from glandular trichome cells, methods to down-regulate at least one molecule endogenous to the plant glandular trichomes can be employed. Trichomes are known to contain a number of compounds and metabolites that interfere with the production of other molecules in the trichome cells. These compounds and metabolites include, for example, proteases, polyphenol oxidase (PPO), polyphenols, ketones, terpenoids, and alkaloids. The down-regulation of such trichome components has been described. See, e.g., U.S. Pat. No. 7,498,428.

### III. Definitions

All technical terms employed in this specification are commonly used in biochemistry, molecular biology and agriculture; hence, they are understood by those skilled in the field to which the present technology belongs. Those technical terms can be found, for example in: Molecular Cloning: A Laboratory Manual 3rd ed., vol. 1-3, ed. Sambrook and Russel (Cold Spring Harbor Laboratory Press, Cold Spring Harbor, N.Y., 2001); Current Protocols In Molecular Biology, ed. Ausubel et al. (Greene Publishing Associates and Wiley-Interscience, New York, 1988) (including periodic updates); Short Protocols In Molecular Biology: A Compendium Of Methods From Current Protocols In Molecular Biology 5th ed., vol. 1-2, ed. Ausubel et al. (John Wiley & Sons, Inc., 2002); Genome Analysis: A Laboratory Manual, vol. 1-2, ed. Green et al. (Cold Spring Harbor Laboratory Press, Cold Spring Harbor, N.Y., 1997). Methodology involving plant biology techniques are described here and also are described in detail in treatises such as Methods In Plant Molecular Biology: A Laboratory Course Manual, ed. Maliga et al. (Cold Spring Harbor Laboratory Press, Cold Spring Harbor, N.Y., 1995).

A “chimeric nucleic acid” comprises a coding sequence or fragment thereof linked to a nucleotide sequence that is different from the nucleotide sequence with which it is associated in cells in which the coding sequence occurs naturally.

The terms “encoding” and “coding” refer to the process by which a gene, through the mechanisms of transcription and translation, provides information to a cell from which a series of amino acids can be assembled into a specific amino acid sequence to produce an active enzyme. Because of the degeneracy of the genetic code, certain base changes in DNA sequence do not change the amino acid sequence of a protein.

“Endogenous nucleic acid” or “endogenous sequence” is “native” to, i.e., indigenous to, the plant or organism that is to be genetically engineered. It refers to a nucleic acid, gene, polynucleotide, DNA, RNA, mRNA, or cDNA molecule that is present in the genome of a plant or organism that is to be genetically engineered.

“Exogenous nucleic acid” refers to a nucleic acid, DNA or RNA, which has been introduced into a cell (or the cell's ancestor) through the efforts of humans. Such exogenous nucleic acid may be a copy of a sequence which is naturally found in the cell into which it was introduced, or fragments thereof

As used herein, “expression” denotes the production of an RNA product through transcription of a gene or the production of the polypeptide product encoded by a nucleotide sequence. “Overexpression” or “up-regulation” is used to indicate that expression of a particular gene sequence or variant thereof, in a cell or plant, including all progeny plants derived thereof, has been increased by genetic engineering, relative to a control cell or plant.

“Genetic engineering” encompasses any methodology for introducing a nucleic acid or specific mutation into a host organism. For example, a plant is genetically engineered when it is transformed with a polynucleotide sequence that suppresses expression of a gene, such that expression of a target gene is reduced compared to a control plant. In the present context, “genetically engineered” includes transgenic plants and plant cells. A genetically engineered plant or plant cell may be the product of any native approach (i.e., involving no foreign nucleotide sequences), implemented by introducing only nucleic acid sequences derived from the host plant species or from a sexually compatible plant species. See, e.g., U.S. Patent Application No. 2004/0107455.

“Heterologous nucleic acid” or “homologous nucleic acid” refer to the relationship between a nucleic acid or amino acid sequence and its host cell or organism, especially in the context of transgenic organisms. A homologous sequence is naturally found in the host species (e.g., a Cannabis plant transformed with a Cannabis gene), while a heterologous sequence is not naturally found in the host cell (e.g., a tobacco plant transformed with a sequence from Cannabis plants). Such heterologous nucleic acids may comprise segments that are a copy of a sequence that is naturally found in the cell into which it has been introduced, or fragments thereof. Depending on the context, the term “homolog” or “homologous” may alternatively refer to sequences which are descendent from a common ancestral sequence (e.g., they may be orthologs).

“Increasing,” “decreasing,” “modulating,” “altering,” or the like refer to comparison to a similar variety, strain, or cell grown under similar conditions but without the modification resulting in the increase, decrease, modulation, or alteration. In some cases, this may be a non-transformed control, a mock transformed control, or a vector-transformed control.

By “isolated nucleic acid molecule” is intended a nucleic acid molecule, DNA, or RNA, which has been removed from its native environment. For example, recombinant DNA molecules contained in a DNA construct are considered isolated for the purposes of the present technology. Further examples of isolated DNA molecules include recombinant DNA molecules maintained in heterologous host cells or DNA molecules that are purified, partially or substantially, in solution. Isolated RNA molecules include in vitro RNA transcripts of the DNA molecules of the present technology. Isolated nucleic acid molecules, according to the present technology, further include such molecules produced synthetically.

“Plant” is a term that encompasses whole plants, plant organs (e.g., leaves, stems, roots, etc.), seeds, differentiated or undifferentiated plant cells, and progeny of the same. Plant material includes without limitation seeds, suspension cultures, embryos, meristematic regions, callus tissues, leaves, roots, shoots, stems, fruit, gametophytes, sporophytes, pollen, and microspores.

“Plant cell culture” means cultures of plant units such as, for example, protoplasts, cell culture cells, cells in plant tissues, pollen, pollen tubes, ovules, embryo sacs, zygotes, and embryos at various stages of development. In some embodiments of the present technology, a transgenic tissue culture or transgenic plant cell culture is provided, wherein the transgenic tissue or cell culture comprises a nucleic acid molecule of the present technology.

“Promoter” connotes a region of DNA upstream from the start of transcription that is involved in recognition and binding of RNA polymerase and other proteins to initiate transcription. A “constitutive promoter” is one that is active throughout the life of the plant and under most environmental conditions. Tissue-specific, tissue-preferred, cell type-specific, and inducible promoters constitute the class of “non-constitutive promoters.” “Operably linked” refers to a functional linkage between a promoter and a second sequence, where the promoter sequence initiates and mediates transcription of the DNA sequence corresponding to the second sequence. In general, “operably linked” means that the nucleic acid sequences being linked are contiguous.

“Sequence identity” or “identity” in the context of two polynucleotide (nucleic acid) or polypeptide sequences includes reference to the residues in the two sequences that are the same when aligned for maximum correspondence over a specified region. When percentage of sequence identity is used in reference to proteins it is recognized that residue positions which are not identical often differ by conservative amino acid substitutions, where amino acid residues are substituted for other amino acid residues with similar chemical properties, such as charge and hydrophobicity, and therefore do not change the functional properties of the molecule. Where sequences differ in conservative substitutions, the percent sequence identity may be adjusted upwards to correct for the conservative nature of the substitution. Sequences which differ by such conservative substitutions are said to have “sequence similarity” or “similarity.” Means for making this adjustment are well-known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, for example, according to the algorithm of Meyers & Miller, Computer Applic. Biol. Sci. 4: 11-17 (1988), as implemented in the program PC/GENE (Intelligenetics, Mountain View, Calif., USA).

Use in this description of a percentage of sequence identity denotes a value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

The terms “suppression” or “down-regulation” are used synonymously to indicate that expression of a particular gene sequence variant thereof, in a cell or plant, including all progeny plants derived thereof, has been reduced by genetic engineering, relative to a control cell or plant.

“Cannabis” or “Cannabis plant” refers to any species in the Cannabis genus that produces cannabinoids, such as Cannabis sativa and interspecific hybrids thereof.

A “variant” is a nucleotide or amino acid sequence that deviates from the standard, or given, nucleotide or amino acid sequence of a particular gene or polypeptide. The terms “isoform,” “isotype,” and “analog” also refer to “variant” forms of a nucleotide or an amino acid sequence. An amino acid sequence that is altered by the addition, removal, or substitution of one or more amino acids, or a change in nucleotide sequence may be considered a variant sequence. A polypeptide variant may have “conservative” changes, wherein a substituted amino acid has similar structural or chemical properties, e.g., replacement of leucine with isoleucine. A polypeptide variant may have “nonconservative” changes, e.g., replacement of a glycine with a tryptophan. Analogous minor variations may also include amino acid deletions or insertions, or both. Guidance in determining which amino acid residues may be substituted, inserted, or deleted may be found using computer programs well known in the art such as Vector NTI Suite (InforMax, MD) software. Variant may also refer to a “shuffled gene” such as those described in Maxygen-assigned patents (see, e.g., U.S. Pat. No. 6,602,986).

As used herein, the term “about” will be understood by persons of ordinary skill in the art and will vary to some extent depending upon the context in which it is used. If there are uses of the term which are not clear to persons of ordinary skill in the art given the context in which it is used, “about” will mean up to plus or minus 10% of the particular term.

The term “biologically active fragments” or “functional fragments” or “fragments having promoter activity” refer to nucleic acid fragments which are capable of conferring transcription in one or more glandular trichomes, one or more trichome cells, vascular tissues and/or cells, and/or one or more different types of plant tissues and organs. In some embodiments, biologically active fragments confer glandular trichome preferred expression, and they preferably have at least a similar strength (or higher strength) as the promoter of SEQ ID NOs: 1, 3, 5, 7, 9, 11, 13, 15, 17, 19, 21, 23, 25, 27, 29, 31, 33, 36, or 39. This can be tested by transforming a plant with such a fragment, preferably operably linked to a reporter gene, and assaying the promoter activity qualitatively (spatio-temporal transcription) and/or quantitatively in trichomes. In some embodiments, the strength of the promoter and/or promoter fragments of the present technology is quantitatively identical to, or higher than, that of the CaMV 35S promoter when measured in the glandular trichome. In some embodiments, a biologically active fragment of a terpene synthase promoter described herein can be about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 91%, about 92%, about 93%, about 94%, about 95%, about 96%, about 97%, about 98%, or about 99% of the full length sequence nucleic acid sequence for the promoter. In other embodiments, a biologically active nucleic acid fragment of a terpene synthase promoter described herein can be, for example, at least about 10 contiguous nucleic acids. In yet other embodiments, the biologically active nucleic acid fragment of a terpene synthase promoter described herein can be (1) about 10 contiguous nucleic acids up to about 1003 contiguous nucleic acids for the TPS1CBDRx promoter (e.g., SEQ ID NO: 1); (2) about 10 contiguous nucleic acids up to about 1003 contiguous nucleic acids for the TPS2CBDRx promoter (SEQ ID NO: 3); (3) about 10 contiguous nucleic acids up to about 1016 contiguous nucleic acids for the TPS3CBDRx promoter (SEQ ID NO: 5); (4) about 10 contiguous nucleic acids up to about 998 contiguous nucleic acids for the TPS4CBDRx promoter (e.g., SEQ ID NO: 7); (5) about 10 contiguous nucleic acids up to about 1037 contiguous nucleic acids for the TPSSCBDRx promoter (e.g., SEQ ID NO: 9); (6) about 10 contiguous nucleic acids up to about 1003 contiguous nucleic acids for the TPS6CBDRx promoter (e.g., SEQ ID NO: 11); (7) about 10 contiguous nucleic acids up to about 1003 contiguous nucleic acids for the TPS7CBDRx promoter (e.g., SEQ ID NO: 13); (8) about 10 contiguous nucleic acids up to about 1003 contiguous nucleic acids for the TPS8CBDRx promoter (e.g., SEQ ID NO: 15); (9) about 10 contiguous nucleic acids up to about 1003 contiguous nucleic acids for the TPS9CBDRx promoter (e.g., SEQ ID NO: 17); (10) about 10 contiguous nucleic acids up to about 1003 contiguous nucleic acids for the TPS10CBDRx promoter (e.g., SEQ ID NO: 19); (11) about 10 contiguous nucleic acids up to about 1091 contiguous nucleic acids for the TPS11CBDRx promoter (e.g., SEQ ID NO: 21); (12) about 10 contiguous nucleic acids up to about 1003 contiguous nucleic acids for the TPS12CBDRx promoter (e.g., SEQ ID NO: 23); (13) about 10 contiguous nucleic acids up to about 1003 contiguous nucleic acids for the TPS13CBDRx promoter (e.g., SEQ ID NO: 25); (14) about 10 contiguous nucleic acids up to about 1003 contiguous nucleic acids for the TPS14CBDRx promoter (e.g., SEQ ID NO: 27); (15) about 10 contiguous nucleic acids up to about 1047 contiguous nucleic acids for the TPS15CBDRx promoter (e.g., SEQ ID NO: 29); (16) about 10 contiguous nucleic acids up to about 1003 contiguous nucleic acids for the TPS16CBDRx promoter (e.g., SEQ ID NO: 31); (17) about 10 contiguous nucleic acids up to about 1071 contiguous nucleic acids for the TPS17CBDRx promoter (e.g., SEQ ID NO: 33); (18) about 10 contiguous nucleic acids up to about 1003 contiguous nucleic acids for the TPS19CBDRx promoter (e.g., SEQ ID NO: 36); or (19) about 10 contiguous nucleic acids up to about 1003 contiguous nucleic acids for the TPS21CBDRx promoter (e.g., SEQ ID NO: 39. In yet other embodiments, the biologically active fragment of the trichome promoter can be any value of contiguous nucleic acids in between these two amounts, such as but not limited to about 20, about 30, about 40, about 50, about 60, about 70, about 80, about 90, about 100, about 110, about 120, about 130, about 140, about 150, about 160, about 170, about 180, about 190, about 200, about 250, about 300, about 350, about 400, about 450, about 500, about 550, about 600, about 650, about 700, about 750, about 800, about 850, about 900, about 950, about 1000, about 1050, about 1100, about 1150, about 1200, about 1250, or about 1300 contiguous nucleic acids.

### EXAMPLES

The following examples are provided by way of illustration only and not by way of limitation. Those of skill in the art will readily recognize a variety of non-critical parameters that could be changed or modified to yield essentially the same or similar results. The examples should in no way be construed as limiting the scope of the present technology, as defined by the appended claims.

### Example 1

### Identifying Terpene Synthase (TPS) Promoters

The SEQ ID NO. for each nucleic acid sequence for each promoter, and the SEQ ID NO. for each corresponding promoter polypeptide, is identified in Table 4 below. The putative enzymatic activities associated with each TPS promoter are provided in Table 1.

TABLE 4
Sequence Identifiers for CBDRx TPS promoter
Nucleic Acid and Amino Acid Sequences.

| | Nucleic Acid Sequence | Amino Acid Sequence of |
|---|---|---|
| Promoter | of the Promoter | the Promoter Polypeptide |
| TPS1CBDRx | SEQ ID NO: 1 | SEQ ID NO: 2 |
| TPS2CBDRx | SEQ ID NO: 3 | SEQ ID NO: 4 |
| TPS3CBDRx | SEQ ID NO: 5 | SEQ ID NO: 6 |
| TPS4CBDRx | SEQ ID NO: 7 | SEQ ID NO: 8 |
| TPS5CBDRx | SEQ ID NO: 9 | SEQ ID NO: 10 |
| TPS6CBDRx | SEQ ID NO: 11 | SEQ ID NO: 12 |
| TPS7CBDRx | SEQ ID NO: 13 | SEQ ID NO: 14 |
| TPS8CBDRx | SEQ ID NO: 15 | SEQ ID NO: 16 |
| TPS9CBDRx | SEQ ID NO: 17 | SEQ ID NO: 18 |
| TPS10CBDRx | SEQ ID NO: 19 | SEQ ID NO: 20 |
| TPS11CBDRx | SEQ ID NO: 21 | SEQ ID NO: 22 |
| TPS12CBDRx | SEQ ID NO: 23 | SEQ ID NO: 24 |
| TPS13CBDRx | SEQ ID NO: 25 | SEQ ID NO: 26 |
| TPS14CBDRx | SEQ ID NO: 27 | SEQ ID NO: 28 |
| TPS15CBDRx | SEQ ID NO: 29 | SEQ ID NO: 30 |
| TPS16CBDRx | SEQ ID NO: 31 | SEQ ID NO: 32 |
| TPS17CBDRx | SEQ ID NO: 33 | SEQ ID NO: 34 |
| TPS18CBDRx | — | SEQ ID NO: 35 |
| TPS19CBDRx | SEQ ID NO: 36 | SEQ ID NO: 37 |
| TPS20CBDRx | — | SEQ ID NO: 38 |
| TPS21CBDRx | SEQ ID NO: 39 | SEQ ID NO: 40 |
| TPS22CBDRx | — | SEQ ID NO: 41 |
| TPS23CBDRx | — | SEQ ID NO: 42 |

The predicted TPS gene sequences in Table 1 were taken and 1,000 bp upstream of the ATG start codon were identified as the promoter in all cases except TPS18CBDRx, where the location of the ATG start codon was unclear and had to be determined experimentally. The 1,000 bp were identified using CoGe (genomevolution.org/coge/), a platform for performing Comparative Genomics research.

### Example 2

### GUS Reporter Construct and Histochemical Staining for β-Glucuronidase

CsTPS1/35PKp and CsTPS4FN promoter sequences were subcloned into ms23 vector carrying reporter gene UidA. After digestion with HindIII and Sac1 restriction enzymes, desired promoter fragments with reporter gene were cloned in the destination binary vector pGPTV. The resulting vector which contains the CsTPS1/35PKp and CsTPS4FN promoter was transformed into Agrobacterium tumefaciens strain GV3101. Generation of transgenic tobacco plants by leaf disc transformation was performed according to Sarowar et al., Plant Cell Reports 24:216-224 (2005). Histochemical analysis of GUS activity was performed using X-gluc (5-bromo-4-chloro-3-indolyl-b-D-glucopyranosiduronic acid) (Gold Biotechnology, St. Louis, Mo.) as the substrate.

As shown in FIGS. 2A and 2B, both promoters direct significant levels of gene expression in tobacco glandular trichomes and the two promoters can therefore be used in methods to manipulate terpene biosynthesis, or the biosynthesis of other biochemicals, in glandular trichomes from plants.

### Example 3

### Identifying Terpene Synthase (TPS) Promoter Consensus Sequences

The nucleic acid sequence of the TPS1U (terpene synthase Glade 1 upstream) conserved promoter domain is set forth in SEQ ID NO: 47. The nucleic acid sequence of the TPS1D (terpene synthase Glade 1 downstream) conserved promoter domain is set forth in SEQ ID NO: 48. The nucleic acid sequence of the TPS3U (terpene synthase Glade 3 upstream) conserved promoter domain is set forth in SEQ ID NO: 49. The nucleic acid sequence of the TPS3D (terpene synthase Glade 3 downstream) conserved promoter domain is set forth in SEQ ID NO: 50.

The consensus sequences of similar TPS promoters were produced using the Pro-Coffee alignment tool that aligns homologous promoter regions. Pro-Coffee is part of the T-Coffee suit of multiple alignment tools (tcoffee.crg.cat/apps/tcoffee/index.html).

### Example 4

### Terpene Synthase (TPS) Promoters and TPS Promoter Consensus Sequences for Directing Terpene Production in Nicotiana tabacum

Terpenes are produced and accumulate in glandular trichomes. Accordingly, it is expected that the promoters for enzymes in the terpene biosynthesis pathway will direct the expression of coding nucleic acids in glandular trichome cells. This example demonstrates the prophetic use of the terpene synthase (TPS) promoters and TPS promoter consensus sequences of the present technology, or biologically active fragments thereof, to modulate the expression of terpene biosynthetic enzymes in tobacco plants.

### Methods

Applicant's tobacco glandular trichome system permits testing of promoters to characterize expression in glandular trichomes and other tissues to provide information regarding the strength of expression of the various promoters.

Vector constructs. TPS promoter sequences and TPS promoter consensus sequences (SEQ ID NOs: 1, 3, 5, 7, 9, 11, 13, 15, 17, 19, 21, 23, 25, 27, 29, 31, 33, 36, 39, 47, 48, 49, or 50) are placed in front of a GUS-A marker in a vector adapted for expression in a Nicotiana tabacum cell, such as a Ti plasmid vector. The constructs can be incorporated into Agrobacterium tumafaciens and used to transform N. tabacum according to methods known in the art. Constructs can be transformed and regenerated under kanamycin selection and primary regenerants (T₀) can be grown to seed.

As a control, a construct containing the tobacco NtCPS2 promoter is transformed into tobacco. The NtCPS2 promoter has been shown to be highly effective in directing trichome expression in N. tabacum (Sallaud et al., The Plant Journal 72:1-17 (2012)).

Expression analysis. Quantitative and qualitative β-glucuronidase (GUS) activity analyses can be performed on T₁ plants. Qualitative analysis of promoter activity can be carried out using histological GUS assays and by visualization of the Green Fluorescent Protein (GFP) using a fluorescence microscope. For GUS assays, various plant parts can be incubated overnight at 37° C. in the presence of atmospheric oxygen with Xglue (5-Bromo-4-chloro-3-indolyl-β-D-glucuronide cyclohexylamine salt) substrate in phosphate buffer (1 mg/mL, K₂HPO₄, 10 μM, pH 7.2, 0.2% Triton X-100). The samples can be de-stained by repeated washing with ethanol. Non-transgenic plants can be used as negative controls. It is anticipated that trichomes of transgenic plants with TPS1CBDRx:GUS, TPS2CBDRx:GUS, TPS3CBDRx:GUS, TPS4CBDRx:GUS, TPS5CBDRx:GUS, TPS6CBDRx:GUS, TPS7CBDRx:GUS, TPS8CBDRx:GUS, TPS9CBDRx:GUS, TPS10CBDRx:GUS, TPS11CBDRx:GUS, TPS12CBDRx:GUS, TPS13CBDRx:GUS, TPS14CBDRx:GUS, TPS15CBDRx:GUS, TPS16CBDRx:GUS, TPS17CBDRx:GUS, TPS18CBDRx:GUS, TPS19CBDRx:GUS, TPS20CBDRx:GUS, TPS21CBDRx:GUS, TPS22CBDRx:GUS, TPS23CBDRx:GUS23, TPS1U:GUS, TPS1D:GUS, TPS3U:GUS, and TPS3D:GUS will show bright blue glandular trichomes with or without expression in other plant tissues whereas the glandular trichomes of control and non-transgenic control plants will not be colored.

Quantitative analysis of promoter activity can be carried out using a fluorometric GUS assay. Total protein samples can be prepared from young leaf material; samples are prepared from pooled leaf pieces. Fresh leaf material is ground in PBS using metal beads followed by centrifugation and collection of the supernatant.

### Results

These results are expected to show that plants genetically engineered with expression vectors comprising the TPS promoters or TPS promoter consensus sequences of the present technology, or biologically active fragments thereof, exhibit strong trichome transcriptional activity. Accordingly, these results are expected to demonstrate that the TPS promoters and TPS promoter consensus sequences as described herein are useful for directing strong expression of an operably linked gene in glandular trichome tissue, as compared to expression in the root, leaf, stem, or other tissues of a plant. This strong trichome expression will be a crucial tool for the manipulation of the biosynthesis of biochemicals in glandular trichomes. In addition, these TPS promoters and TPS promoter consensus sequences will be crucial to strategies aimed at using tobacco glandular trichomes as biofactories for the controlled production of specific biochemical compounds, including terpenes.

### Example 5

### Terpene Synthase (TPS) Promoters and TPS Promoter Consensus Sequences for Directing Terpene Production in Cannabis sativa

Terpenes are produced and accumulate in Cannabis glandular trichomes. Accordingly, it is expected that the promoters for enzymes in the terpene biosynthesis pathway will direct the expression of coding nucleic acids in glandular trichome cells. This prophetic example demonstrates the use of the terpene synthase (TPS) promoters and TPS promoter consensus sequences of the present technology, or biologically active fragments thereof, to modulate the expression of terpene biosynthetic enzymes in Cannabis.

### Methods

Vector constructs. TPS promoter sequences and TPS promoter consensus sequences (SEQ ID NOs: 1, 3, 5, 7, 9, 11, 13, 15, 17, 19, 21, 23, 25, 27, 29, 31, 33, 36, 39, 47, 48, 49, or 50) are placed in front of a GUS-A marker in a vector adapted for expression in a Cannabis sativa cell. The constructs can be incorporated into Agrobacterium tumafaciens and used to transform C. sativa. Constructs can be transformed and regenerated under kanamycin selection and primary regenerants (T₀) can be grown to seed.

As a control, a construct containing a promoter effective at directing trichome expression in C. sativa can be transformed into control C. sativa cells.

Expression analysis. Quantitative and qualitative β-glucuronidase (GUS) activity analyses can be performed on T₁ plants. Qualitative analysis of promoter activity can be carried out using histological GUS assays and by visualization of the Green Fluorescent Protein (GFP) using a fluorescence microscope. For GUS assays, various plant parts can be incubated overnight at 37° C. in the presence of atmospheric oxygen with Xglue (5-Bromo-4-chloro-3-indolyl-(3-D-glucuronide cyclohexylamine salt) substrate in phosphate buffer (1 mg/mL, K₂HPO₄, 10 μM, pH 7.2, 0.2% Triton X-100). The samples can be de-stained by repeated washing with ethanol. Non-transgenic plants are used as negative controls. It is anticipated that trichomes of transgenic plants with TPS1CBDRx:GUS, TPS2CBDRx:GUS, TPS3CBDRx:GUS, TPS4CBDRx:GUS, TPS5CBDRx:GUS, TPS6CBDRx:GUS, TPS7CBDRx:GUS, TPS8CBDRx:GUS, TPS9CBDRx:GUS, TPS10CBDRx:GUS, TPS11CBDRx:GUS, TPS12CBDRx:GUS, TPS13CBDRx:GUS, TPS14CBDRx:GUS, TPS15CBDRx:GUS, TPS16CBDRx:GUS, TPS17CBDRx:GUS, TPS18CBDRx:GUS, TPS19CBDRx:GUS, TPS20CBDRx:GUS, TPS21CBDRx:GUS, TPS22CBDRx:GUS, TPS23CBDRx:GUS23, TPS1U:GUS, TPS1D:GUS, TPS3U:GUS, and TPS3D:GUS will show bright blue glandular trichomes with or without expression in other plant tissues whereas the glandular trichomes of control and non-transgenic control plants will not be colored.

Quantitative analysis of promoter activity can be carried out using a fluorometric GUS assay. Total protein samples can be prepared from young leaf material; samples are prepared from pooled leaf pieces. Fresh leaf material is ground in PBS using metal beads followed by centrifugation and collection of the supernatant.

### Results

These results are expected to show that plants genetically engineered with expression vectors comprising the TPS promoters or TPS promoter consensus sequences of the present technology, or biologically active fragments thereof, exhibit strong trichome transcriptional activity. Accordingly, these results are expected to demonstrate that the TPS promoters and TPS promoter consensus sequences as described herein are useful for directing strong expression of an operably linked gene in glandular trichome tissue, as compared to expression in the root, leaf, stem, or other tissues of a plant. This strong trichome expression will be a crucial tool for the manipulation of the biosynthesis of biochemicals in glandular trichomes. In addition, these TPS promoters and TPS promoter consensus sequences will be crucial to strategies aimed at using Cannabis glandular trichomes as biofactories for the controlled production of specific biochemical compounds, including terpenes.

### REFERENCES

Jones D. T., Taylor W. R., and Thornton J. M. (1992). The rapid generation of mutation data matrices from protein sequences. Computer Applications in the Biosciences 8: 275-282.
Tamura K., Stecher G., Peterson D., Filipski A., and Kumar S. (2013). MEGA6: Molecular Evolutionary Genetics Analysis version 6.0. Molecular Biology and Evolution30: 2725-2729.
Saitou N. and Nei M. (1987). The neighbor-joining method: A new method for reconstructing phylogenetic trees. Molecular Biology and Evolution 4:406-425.
Christopher J Grassa, Jonathan P Wenger, Clemon Dabney, Shane G Poplawski, S Timothy Motley, Todd P Michael, C J Schwartz, George D Weiblen (2018). A complete Cannabis chromosome assembly and adaptive admixture for elevated cannabidiol (CBD) content. bioRxiv 458083.
Judith K. Booth, Jonathan E. Page, and Jörg Bohlmann (2017). Terpene synthases from Cannabis sativa.

### EQUIVALENTS

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the appended claims. The present technology is to be limited only by the terms of the appended claims, along with the full scope of equivalents to which such claims are entitled. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

All publicly available documents referenced or cited to herein, such as patents, patent applications, provisional applications, and publications, including GenBank Accession Numbers, are incorporated by reference in their entirety, including all figures and tables, to the extent they are not inconsistent with the explicit teachings of this specification.

Other embodiments are set forth within the following claims.

1. A synthetic DNA molecule comprising a nucleotide sequence selected from the group consisting of:
(a) a nucleotide sequence set forth in any one of SEQ ID NOs: 1, 3, 5, 7, 9, 11, 13, 15, 17, 19, 21, 23, 25, 27, 29, 31, 33, 36, 39, 47, 48, 49, or 50;
(b) a nucleotide sequence that encodes for a polypeptide having the amino acid sequence of any one of SEQ ID NOs: 2, 4, 6, 8, 10, 12, 14, 16, 18, 20, 22, 24, 26, 28, 30, 32, 34, 35, 37, 38, 40, 41, or 42; and
(c) a nucleotide sequence that is at least about 80% identical to the nucleotide sequence of (a) or (b), and which encodes a promoter having plant glandular trichome transcriptional activity,
wherein the nucleotide sequence is operably linked to a heterologous nucleic acid.

2. An expression vector comprising the DNA molecule of claim 1, operably linked to one or more nucleic acid sequences encoding a polypeptide.

3. A genetically engineered host cell comprising the expression vector of claim 2.

4. The genetically engineered host cell of claim 3, wherein:
(a) the cell is from a plant having glandular trichomes; and/or
(b) the cell is a Cannabis sativa cell; and/or
(c) the cell is a Nicotiana tabacum cell.

5. (canceled)

6. (canceled)

7. A genetically engineered plant comprising a cell comprising a chimeric nucleic acid construct comprising the synthetic DNA molecule of claim 1.

8. The engineered plant of claim 7, wherein:
(a) the plant contains glandular trichomes; and/or
(b) the plant is an Nicotiana tabacum plant and/or
(c) the plant is a Cannabis sativa plant.

9. (canceled)

10. (canceled)

11. Seeds from the engineered plant of claim 1, wherein the seeds comprise the chimeric nucleic acid construct.

12. A genetically engineered plant or plant cell comprising a chimeric gene integrated into its genome, the chimeric gene comprising a terpene synthase (TPS) promoter operably linked to a homologous or heterologous nucleic acid sequence, wherein the promoter is selected from the group consisting of:
(a) a nucleotide sequence of any one of SEQ ID NOs: 1, 3, 5, 7, 9, 11, 13, 15, 17, 19, 21, 23, 25, 27, 29, 31, 33, 36, 39, 47, 48, 49, or 50;
(b) a nucleotide sequence that encodes for a polypeptide having the amino acid sequence of any one of SEQ ID NOs: 2, 4, 6, 8, 10, 12, 14, 16, 18, 20, 22, 24, 26, 28, 30, 32, 34, 35, 37, 38, 40, 41, or 42; and
(c) a nucleotide sequence that is at least about 80% identical to the nucleotide sequence of (a) or (b), and which encodes a promoter that has plant glandular trichome transcriptional activity.

13. The genetically engineered plant or plant cell of claim 12, wherein:
(a) the plant contains glandular trichomes; and/or
(b) the plant is an Nicotiana tabacum plant; and/or
(c) the plant is a Cannabis sativa plant.

14. (canceled)

15. (canceled)

16. A method for expressing a polypeptide in plant trichomes, comprising:
(a) introducing into a host cell an expression vector comprising a nucleotide sequence selected from the group consisting of:
a nucleotide sequence set forth in any one of SEQ ID NOs: 1, 3, 5, 7, 9, 11, 13, 15, 17, 19, 21, 23, 25, 27, 29, 31, 33, 36, 39, 47, 48, 49, or 50;
(ii) a nucleotide sequence that encodes for a polypeptide having the amino acid sequence of any one of SEQ ID NOs: 2, 4, 6, 8, 10, 12, 14, 16, 18, 20, 22, 24, 26, 28, 30, 32, 34, 35, 37, 38, 40, 41, or 42; and
(iii) a nucleotide sequence that is at least about 80% identical to the nucleotide sequence of (a) or (b), and which encodes a promoter that has plant glandular trichome transcriptional activity;
wherein the nucleic acid sequence of (i) or (ii) is operably linked to one or more nucleic acid sequences encoding a polypeptide; and
(b) growing the plant under conditions which allow for the expression of the polypeptide.

17. A method for increasing a terpene in a host plant glandular trichome, comprising:
(a) introducing into a host cell an expression vector comprising a nucleotide sequence selected from the group consisting of:
a nucleotide sequence set forth in any one of SEQ ID NOs: 1, 3, 5, 7, 9, 11, 13, 15, 17, 19, 21, 23, 25, 27, 29, 31, 33, 36, 39, 47, 48, 49, or 50;
(ii) a nucleotide sequence that encodes for a polypeptide having the amino acid sequence of any one of SEQ ID NOs: 2, 4, 6, 8, 10, 12, 14, 16, 18, 20, 22, 24, 26, 28, 30, 32, 34, 35, 37, 38, 40, 41, or 42; and
(iii) a nucleotide sequence that is at least about 80% identical to the nucleotide sequence of (a) or (b), and which encodes a promoter that has plant glandular trichome transcriptional activity;
wherein the nucleic acid sequence of (i) or (ii) is operably linked to one or more nucleic acid sequences encoding an enzyme of the terpene biosynthetic pathway; and
(b) growing the plant under conditions which allow for the expression of the terpene biosynthetic pathway enzyme;
wherein expression of the terpene biosynthetic pathway enzyme results in the plant having an increased terpene content relative to a control plant grown under similar conditions.

18. The method of claim 17, wherein the terpene biosynthetic pathway enzyme is limonene synthase, squalene synthase, phytoene synthase, myrcene synthase, germacrene D synthase, α-farnesene synthase, or geranyllinalool synthase.

19. The method of claim 18, further comprising providing the plant with isopentenyl diphosphate (IPP), dimethyl allyl diphosphate (DMAPP), or geranyl pyrophosphate (GPP).

20. A genetically-engineered plant produced by the method of claim 17, wherein the plant has increased terpene content relative to a control plant.

21. A genetically engineered plant or plant cell comprising a chimeric gene integrated into its genome, the chimeric gene comprising a terpene synthase (TPS) promoter operably linked to a homologous or heterologous nucleic acid sequence, wherein the promoter is selected from the group consisting of:
(a) a nucleotide sequence of any one of SEQ ID NOs: 44 or 46;
(b) a nucleotide sequence that encodes for a polypeptide having the amino acid sequence of any one of SEQ ID NOs: 43 or 45; and
(c) a nucleotide sequence that is at least about 80% identical to the nucleotide sequence of (a) or (b), and which encodes a promoter that has plant glandular trichome transcriptional activity.

22. The genetically engineered plant or plant cell of claim 21, wherein:
(a) the plant contains glandular trichomes; and/or
(b) the plant is an Nicotiana tabacum plant; and/or
(c) the plant is a Cannabis sativa plant.

23. (canceled)

24. (canceled)

25. A method for expressing a polypeptide in plant trichomes, comprising:
(a) introducing into a host cell an expression vector comprising a nucleotide sequence selected from the group consisting of:
(i) a nucleotide sequence set forth in any one of SEQ ID NOs:44 or 46;
(ii) a nucleotide sequence that encodes for a polypeptide having the amino acid sequence of any one of SEQ ID NOs: 43 or 45; and
(iii) a nucleotide sequence that is at least about 80% identical to the nucleotide sequence of (a) or (b), and which encodes a promoter that has plant glandular trichome transcriptional activity;
wherein the nucleic acid sequence of (i) or (ii) is operably linked to one or more nucleic acid sequences encoding a polypeptide; and
(b) growing the plant under conditions which allow for the expression of the polypeptide.

26. A method for increasing a terpene in a host plant glandular trichome, comprising:
(a) introducing into a host cell an expression vector comprising a nucleotide sequence selected from the group consisting of:
(i) a nucleotide sequence set forth in any one of SEQ ID NOs: 44 or 46;
(ii) a nucleotide sequence that encodes for a polypeptide having the amino acid sequence of any one of SEQ ID NOs: 43 or 45; and
(iii) a nucleotide sequence that is at least about 80% identical to the nucleotide sequence of (a) or (b), and which encodes a promoter that has plant glandular trichome transcriptional activity;
wherein the nucleic acid sequence of (i) or (ii) is operably linked to one or more nucleic acid sequences encoding an enzyme of the terpene biosynthetic pathway; and
(b) growing the plant under conditions which allow for the expression of the terpene biosynthetic pathway enzyme;
wherein expression of the terpene biosynthetic pathway enzyme results in the plant having an increased terpene content relative to a control plant grown under similar conditions.

27. The method of claim 26, wherein the terpene biosynthetic pathway enzyme is limonene synthase, squalene synthase, phytoene synthase, myrcene synthase, germacrene D synthase, α-farnesene synthase, or geranyllinalool synthase.

28. The method of claim 27, further comprising providing the plant with isopentenyl diphosphate (IPP), dimethyl allyl diphosphate (DMAPP), or geranyl pyrophosphate (GPP).

29. A genetically-engineered plant produced by the method of claim 26, wherein the plant has increased terpene content relative to a control plant.
